(19) United States
(12) Patent Application Publication
(10) Pub. No.: US 2025/0327124 A1
(43) Pub. Date: Oct. 23, 2025

(54) METHOD FOR TESTING PULMONARY HYPERTENSION, PATHOLOGICAL ANIMAL MODEL FOR PULMONARY ARTERIAL HYPERTENSION, AND PREVENTIVE OR THERAPEUTIC AGENT FOR PULMONARY HYPERTENSION

(71) Applicant: KYOTO UNIVERSITY, Kyoto-shi, Kyoto (JP)

(72) Inventors: Yoshikazu NAKAOKA, Suita-shi (JP); Osamu TAKEUCHI, Kyoto-shi (JP); Ai YAKU, Kyoto-shi (JP)

(73) Assignee: KYOTO UNIVERSITY, Kyoto-shi, Kyoto (JP)

(21) Appl. No.: 18/711,335

(22) PCT Filed: Nov. 17, 2022
(86) PCT No.: PCT/JP2022/042690

§ 371 Date: Apr. 4, 2025

(30) Foreign Application Priority Data
Nov. 17, 2021 (JP) 2021-187370

(51) Int. Cl. C12Q 1/6883; A61P 9/12; C12N 15/113; G01N 33/573

(52) U.S. Cl. CPC C12Q 1/6883; A61P 9/12; C12N 15/1137; G01N 33/573; C12N 2310/113; C12N 2310/14; C12Q 2600/158; G01N 2333/916

(57) ABSTRACT

A purpose of the present disclosure is to provide a method for testing presence/absence, severity or prognosis of pulmonary hypertension; a pathological model animal for pulmonary arterial hypertension; and a prophylactic or therapeutic drug for hypertension. Provided are: a testing method for hypertension, with Regnase-1 used as a biomarker; a PAH pathological model animal consisting of a non-human animal with Regnase-1 deficiency in alveolar macrophage; and a prophylactic or therapeutic drug for hypertension, containing a substance that disrupts a stem-loop structure in 3′UTR of Regnase-1 mRNA.

### TECHNICAL FIELD

The present disclosure relates to a method and an agent for testing presence/absence, severity, or prognosis of pulmonary hypertension. The present disclosure also relates to a pathological model animal for pulmonary arterial hypertension; a method for screening a prophylactic or therapeutic drug for pulmonary arterial hypertension with use of the model animal; and a method for assessing medicinal effect for pulmonary arterial hypertension. The present disclosure further relates to a prophylactic or therapeutic drug for pulmonary hypertension.

### BACKGROUND ART

Pulmonary hypertension (PH) is a progressive disease group with poor prognosis, which causes cardiac and pulmonary dysfunction due to high blood pressure in the pulmonary artery. The Nice Classification classifies PH into Group 1: pulmonary arterial hypertension (PAH), Group 1′: pulmonary atresia (pulmonary veno-occlusive disease, PVOD) and/or pulmonary capillary hemangiomatosis (PCH), Group 1″: neonatal protraction pulmonary hypertension, Group 2: pulmonary hypertension associated with left heart disease, Group 3: pulmonary hypertension associated with pulmonary disease and/or hypoxemia, Group 4: chronic thromboembolic pulmonary hypertension (CTEPH), and Group 5: pulmonary hypertension associated with multifactorial mechanism with unknown details.

Among PH, PAH is a disease having a main locus of inflammation in peripheral pulmonary arteries (small arteries or arterioles), accompanied by pathological changes such as excessive surrounding with vascular smooth muscle, neointimal proliferation, and plexiform lesions of pulmonary artery (NPLs 1 and 2). PAH is further subclassified into idiopathic pulmonary arterial hypertension (idiopathic PAH; IPAH), heritable pulmonary arterial hypertension (heritable PAH; HPAH), drug- or toxin-induced pulmonary arterial hypertension, and pulmonary arterial hypertension associated with various diseases (associated PAH). PAH associated with various diseases is subdivided into connective tissue disease (CTD), human immunodeficiency virus (HIV) infection, portal hypertension, congenital heart disease, and schistosomiasis (Japanese Circulation Society Guidelines: https://www.j-circ.or.jp/cms/wp-content/uploads/2020/02/JCS2017_fukuda_h.pdf).

The etiology of PAH is complex, supposedly ascribed to combination of a variety of factors such as genetic background, epigenetic modification factors, existing diseases, and environmental factors (NPL 3), to which also growth factors and cytokines are considered to make great contribution. The growth factors possibly involved in PAH include platelet-derived growth factor (PDGF), vascular endothelial growth factor (VEGF), and bone morphogenetic proteins (BMPs). Circulating inflammatory cytokines such as Il-6, Il-1β, and tumor necrosis factor (TNF) are also known to increase in PAH patients. Also animal studies have demonstrated that cytokines contribute to the pathogenesis of PAH (NPL 4). These cytokines have also be known to be controlled by factors such as aryl hydrocarbon receptor which plays an important role in onset of PAH (NPL 5).

Innate immune cells have widely been known as major producing cells of inflammatory cytokines in infectious diseases, and in fact, also the innate immune cell such as macrophage has also been known to play an important role in inducing PAH. Inflammatory response of the macrophage is regulated by transcriptional and post-transcriptional mechanisms (NPL 6), and epigenetic dysregulation and miRNAs have been found to be important etiologies of PAH. Various RNA binding proteins (RBPs) that regulate translation and degradation of mRNA involved in immune response have been known to take part in post-transcriptional regulation of immune response (NPL 7). Importance of RBPs in PAH has, however, remained almost unknown.

Meanwhile, Regnase-1 (a protein encoded by the ZC3H12A gene) is an RNA-binding protein that plays an important role in post-transcriptional immunoregulation of innate and acquired immune cells (NPLs 8 and 9). Regnase-1 has an RNase activity that involves recognition of a stem-loop structure in the 3′ untranslated region (3′UTR), and decomposition of mRNA involved in an immune response such as 11-6 and 11-10 (NPL 10). Decomposition of mRNA mediated by Regnase-1 is initiated in a helicase UPF1-dependent manner, after the end of translation (NPL 11). Regnase-1 regulates various immune cells (NPL 12). Deficiency of Regnase-1 in mice has been known to induce severe autoimmune inflammatory disease. Mice with Regnase-1 deficiency in cardiomyocyte has been known to develop heart failure, under pressure load due to transverse aortic coarctation (NPL 13). Regnase-1 has also been reported to be involved in diseases such as ulcerative colitis and pulmonary fibrosis (NPLs 14 to 16). Relationship between PH and Regnase-1 has, however, remained unknown.

### CITATION LIST

### Non Patent Literatures

NPL 1: Humbert M, Sitbon O, Simonneau G. (2004). Treatment of pulmonary arterial hypertension. The New England journal of medicine, 351, 1425-1436.
NPL: 2 Rabinovitch M. (2012). Molecular pathogenesis of pulmonary arterial hypertension. The Journal of clinical investigation, 122, 4306-4313.
NPL 3: Humbert M, Guignabert C, Bonnet S, et al. (2019). Pathology and pathobiology of pulmonary hypertension: state of the art and research perspectives. The European respiratory journal, 53(1): 1801887
NPL 4: Hashimoto-Kataoka T, Hosen N, Sonobe T, et al. (2015). Interleukin-6/interleukin-21 signaling axis is critical in the pathogenesis of pulmonary arterial hypertension. Proceedings of the National Academy of Sciences of the United States of America, 112, E2677-86.
NPL 5: Masaki T, Okazawa M, Asano R, et al. Aryl hydrocarbon receptor is essential for the pathogenesis of pulmonary arterial hypertension. Proc Natl Acad Sci USA. 118(11): e2023899118, 2021.
NPL 6: Takeuchi, O., and Akira, S. (2010). Pattern recognition receptors and inflammation. Cell 140, 805-820.
NPL 7: Carpenter, S., Ricci, E. P., Mercier, B. C., Moore, M. J., and Fitzgerald, K. A. (2014). Post-transcriptional regulation of gene expression in innate immunity. Nat Rev Immunol 14, 361-376.
NPL 8: Mino, T., and Takeuchi, O. (2021). Regnase-1-related endoribonucleases in health and immunological diseases. Immunol Rev. 2021 Sep. 12. doi: 10.1111/imr.13023. Online ahead of print.
NPL 9: Matsushita, K., Takeuchi, O., Standley, D. M., Kumagai, Y., Kawagoe, T., Miyake, T., Satoh, T., Kato, H., Tsujimura, T., Nakamura, H., et al. (2009). Zc3h12a is an RNase essential for controlling immune responses by regulating mRNA decay. Nature 458, 1185-1190.
NPL 10: Mino, T., Murakawa, Y., Fukao, A., Vandenbon, A., Wessels, H. H., Ori, D., Uehata, T., Tartey, S., Akira, S., Suzuki, Y., et al. (2015). Regnase-1 and Roquin Regulate a Common Element in Inflammatory mRNAs by Spatiotemporally Distinct Mechanisms. Cell 161, 1058-1073.
NPL 11: Mino, T., Iwai, N., Endo, M., Inoue, K., Akaki, K., Hia, F., Uehata, T., Emura, T., Hidaka, K., Suzuki, Y., et al. (2019). Translation-dependent unwinding of stem-loops by UPF1 licenses Regnase-1 to degrade inflammatory mRNAs. Nucleic Acids Res 47, 8838-8859.
NPL 12: Uehata, T., Iwasaki, H., Vandenbon, A., Matsushita, K., Hemandez-Cuellar, E., Kuniyoshi, K., Satoh, T., Mino, T., Suzuki, Y., Standley, D. M., et al. (2013). Maltl-induced cleavage of Regnase-1 in CD4(+) helper T cells regulates immune activation. Cell 153, 1036-1049.
NPL 13: Omiya, S., Omori, Y., Taneike, M., Murakawa, T., Ito, J., Tanada, Y., Nishida, K., Yamaguchi, O., Satoh, T., Shah, A. M., et al. (2020). Cytokine mRNA Degradation in Cardiomyocytes Restrains Sterile Inflammation in Pressure-Overloaded Hearts. Circulation 141, 667-677.
NPL 14: Kakiuchi, N., Yoshida, K., Uchino, M., Kihara, T., Akaki, K., Inoue, Y., Kawada, K., Nagayama, S., Yokoyama, A., Yamamoto, S., et al. (2020). Frequent mutations that converge on the NFKBIZ pathway in ulcerative colitis. Nature 577, 260-265.
NPL 15: Nanki, K., Fujii, M., Shimokawa, M., Matano, M., Nishikori, S., Date, S., Takano, A., Toshimitsu, K., Ohta, Y., Takahashi, S., et al. (2020). Somatic inflammatory gene mutations in human ulcerative colitis epithelium. Nature 577, 254-259.
NPL 16: Nakatsuka, Y., Yaku, A., Handa, T., Vandenbon, A., Hikichi, Y., Motomura, Y., Sato, A., Yoshinaga, M., Tanizawa, K., Watanabe, K., et al. (2021). Profibrotic function of pulmonary group 2 innate lymphoid cells is controlled by Regnase-1. Eur Respir J 57(3):2000018.

### SUMMARY OF INVENTION

### Technical Problem

It is therefore an object of the present disclosure to provide a method for identifying a marker that serves as an index of presence/absence, severity, and prognosis prediction of PH, and for testing the presence/absence, the severity, and the prognosis of PH with use of the marker.

Another object of the present disclosure is to provide a model animal that spontaneously develops PAH, a method for screening a prophylactic or therapeutic drug for PAH with use of the model animal, and a method for assessing medicinal effect of a test substance with use of the model animal.

Yet another object of the present disclosure is to provide a prophylactic or therapeutic drug for PH.

### Solution to Problem

The present inventors have found from our comparative study by qPCR, on Regnase-1 mRNA level in human peripheral blood mononuclear cell (PBMC) between PH patients and healthy subjects, that the PH patients demonstrated significant decrease in the Regnase-1 level as compared with the healthy subjects; that the lesser the Regnase-1 level in PMBC, the severer the PH; and that the lesser the Regnase-1 level in PBMC, the poorer the PH prognosis, and found out that Regnase-1 is usable as a PH marker.

Meanwhile, prior creation of a PAH pathological model animal has required hypoxia load and exposure to chemical substances such as monocrotaline and Sugen 5416. The present inventors have found that a mouse, with Regnase-1 knocked out in immune cells thereof, particularly in alveolar macrophage, spontaneously develops PAH without hypoxia load, chemical substance exposure or the like, and is therefore usable as a PAH pathological model mouse. The present inventors have also found that use of the PAH disease model mouse enables screening of a prophylactic or therapeutic drug for PAH, and assessment of medicinal effect of a test substance for PAH.

Furthermore, the present inventors have found that the PH pathological model mouse improved the clinical condition of PH, as a result of administration of morpholino oligo that targets a stem-loop forming sequence in the 3′UTR of Regnase-1 mRNA, so as to disrupt the stem-loop structure in the 3′UTR of Regnase-1 mRNA.

The present disclosure has been completed by further conducting studies on the basis of these findings. That is, the present disclosure exemplifies embodiments with modes below.

Item 1-1. A method for testing presence/absence, severity, or prognosis of pulmonary hypertension, the test method including:
measuring Regnase-1 gene expression level or Regnase-1 level in a sample isolated from a subject.

Item 1-2. The method according to Item 1-1, wherein the pulmonary hypertension is pulmonary arterial hypertension.

Item 1-3. The method according to Item 1-1 or 1-2, wherein the pulmonary hypertension is idiopathic pulmonary arterial hypertension, heritable pulmonary arterial hypertension, or pulmonary arterial hypertension associated with connective tissue disease.

Item 1-4. The method according to Item 1-1 or 1-2, wherein the pulmonary hypertension is idiopathic pulmonary arterial hypertension.

Item 1-5. The method according to Item 1-1 or 1-2, wherein the pulmonary hypertension is heritable pulmonary arterial hypertension.

Item 1-6. The method according to Item 1-1 or 1-2, wherein the pulmonary hypertension is pulmonary arterial hypertension associated with connective tissue disease.

Item 1-7. A test kit for testing presence/absence, severity, or prognosis of pulmonary hypertension, the test kit including a reagent for measuring Regnase-1 gene expression level or Regnase-1 level.

Item 1-8. A reagent used for measuring Regnase-1 gene expression level or Regnase-1 level, for use in a test of presence/absence, severity, or prognosis of pulmonary hypertension.

Item 1-9. Use of a reagent for measuring Regnase-1 gene expression level or Regnase-1 level, for the manufacture of an agent for testing presence/absence, severity, or prognosis of pulmonary hypertension.

Item 2-1. A pathological model animal for pulmonary arterial hypertension, including a non-human animal with Regnase-1 deficiency in at least one immune cell.

Item 2-2. The pathological model animal for pulmonary arterial hypertension according to Item 2-1, wherein the immune cell includes at least an alveolar macrophage.

Item 2-3. A method for screening a candidate substance that is applicable to a prophylactic or therapeutic drug for pulmonary arterial hypertension from among test substances, the screening method including:
a first step of administrating a test substance to a non-human animal with Regnase-1 deficiency in at least one immune cell;
a second step of examining a clinical condition of pulmonary arterial hypertension of the non-human animal with administration of the test substance; and
a third step of selecting the test substance as a candidate substance that can be used as a prophylactic or therapeutic drug for pulmonary arterial hypertension, if the clinical condition of pulmonary arterial hypertension in the non-human animal with administration of the test substance is found to improve as compared with that in the non-human animal without administration of the test substance.

Item 2-4. A method for assessing medicinal effect of a test substance on pulmonary arterial hypertension, the medicinal effect assessing method including:
step I of administering a test substance to a non-human animal with Regnase-1 deficiency in at least one immune cell; and
step II of examining a clinical condition of pulmonary arterial hypertension in the non-human animal with administration of the test substance.

Item 3-1. A prophylactic or therapeutic drug for pulmonary hypertension, containing a substance that disrupts a stem-loop structure in 3′UTR of Regnase-1 mRNA.

Item 3-2. The prophylactic or therapeutic drug for pulmonary hypertension according to Item 3-1, wherein the substance is an oligonucleic acid that hybridizes to at least a part of a base sequence of a region that forms a stem portion of the stem-loop structure in 3′UTR of Regnase-1 mRNA, and inhibits complementary binding in the stem-loop structure.

Item 3-3. The prophylactic or therapeutic drug for pulmonary hypertension according to Item 3-1 or 3-2, wherein the substance is at least one oligonucleic acid selected from the group consisting of (a-1), (a-2), (b-1), and (b-2) below:
(a-1) an oligonucleic acid 10 to 30 bases in length that hybridizes to a sequence having 10 to 30 consecutive bases including positions 233 to 235 in SEQ ID NO: 1, within a base sequence from positions 206 to 242 in SEQ ID NO: 1;
(a-2) an oligonucleic acid 10 to 30 bases in length that hybridizes to a sequence having 10 to 30 consecutive bases including positions 241 to 243 in SEQ ID NO: 1, within a base sequence from positions 234 to 270 in SEQ ID NO: 1;
(b-1) an oligonucleic acid 10 to 30 bases in length that hybridizes to a sequence having 10 to 30 consecutive bases including positions 426 to 428 in SEQ ID NO: 1, within a base sequence from positions 399 to 439 in SEQ ID NO: 1; and
(b-2) an oligonucleic acid 10 to 30 bases in length that hybridizes to a sequence having 10 to 30 consecutive bases including positions 438 to 440 in SEQ ID NO: 1, within a base sequence from positions 427 to 467 in SEQ ID NO: 1.

Item 3-4. The prophylactic or therapeutic drug for pulmonary hypertension according to Item 3-3, wherein the substance includes a combination of at least one oligonucleic acid selected from the group consisting of (a-1) and (a-2), and at least one oligonucleic acid selected from the group consisting of (b-1) and (b-2).

Item 3-5. The prophylactic or therapeutic drug for pulmonary hypertension according to any one of Items 3-1 to 3-4, wherein the pulmonary hypertension is pulmonary arterial hypertension.

Item 3-6. The prophylactic or therapeutic drug for pulmonary hypertension according to any one of Items 3-1 to 3-5, wherein the pulmonary hypertension is idiopathic pulmonary arterial hypertension, heritable pulmonary arterial hypertension, or pulmonary arterial hypertension associated with connective tissue disease.

Item 3-7. The prophylactic or therapeutic drug for pulmonary hypertension according to any one of Items 3-1 to 3-5, wherein the pulmonary hypertension is idiopathic pulmonary arterial hypertension.

Item 3-8. The prophylactic or therapeutic drug for pulmonary hypertension according to any one of Items 3-1 to 3-5, wherein the pulmonary hypertension is heritable pulmonary arterial hypertension.

Item 3-9. The prophylactic or therapeutic drug for pulmonary hypertension according to any one of Items 3-1 to 3-5, wherein the pulmonary hypertension is pulmonary arterial hypertension associated with connective tissue disease.

Item 3-10. A method for preventing or treating pulmonary hypertension, the method comprising a step of administering a substance that disrupts a stem-loop structure in 3′UTR of Regnase-1 mRNA, to a pulmonary hypertension patient or a person at risk of recurrence of pulmonary hypertension.

Item 3-11. A substance that disrupts a stem-loop structure in 3′UTR of Regnase-1 mRNA, for use in prevention or treatment of pulmonary hypertension.

Item 3-12. Use of a substance that disrupts a stem-loop structure in 3′UTR of Regnase-1 mRNA, for use in manufacture of a prophylactic or therapeutic drug for pulmonary hypertension.

### Advantageous Effects of Invention

The PH test method of the present disclosure can test presence/absence of contract with PH, severity of PH, and prognosis of PH, with use of Regnase-1 as a PH marker.

The PAH pathological model animal of the present disclosure, which is a non-human animal with Regnase-1 knocked out in at least one immune cell, can spontaneously develop PAH without hypoxia load, chemical substance exposure, or the like.

According to the method for screening a candidate substance that can be a prophylactic or therapeutic drug for PAH of the present disclosure, it is possible to determine the clinical usefulness of the candidate substance as a prophylactic or therapeutic drug for PAH, without undergoing a step of subjecting a non-human animal to hypoxia load, chemical substance exposure, or the like.

The method for assessing medicinal effect for PAH of the present disclosure can assess the medicinal effect of a candidate substance for PAH, without subjecting a non-human animal to hypoxia load, chemical substance exposure, or the like.

The prophylactic or therapeutic drug for PH of the present disclosure can suppress onset of PH, or can improve clinical condition of PH, by decomposing the stem-loop structure in the 3′UTR of Regnase-1 mRNA, thereby suppressing decrease in expression level of Regnase-1.

### BRIEF DESCRIPTION OF DRAWINGS

FIG. 1(a) is a diagram illustrating Regnase-1 expression level in peripheral blood mononuclear cell (PBMC) of PH patients (n=77) and healthy volunteers (HV) (n=77). FIG. 1(b) is a diagram illustrating Regnase-1 expression level in PBMC of PH patients (n=77) divided into two groups of mild group (classes 1 and 2) (n=32), and moderate-to-severe group (classes 3 and 4) (n=45) in terms of severity according to the WHO functional classification (WHO-FC). FIG. 1(c) is a diagram illustrating event-free survival of PH patients (n=17) divided into high group (n=10) and low group (n=7) in terms of Regnase-1 expression level in PBMC, with the end point defined by death, lung transplantation, and heart failure. FIG. 1(d) is a diagram illustrating relationship between Regnase-1 expression level in PBMC and mean pulmonary arterial pressure (mPAP) of PH patients (n=77). FIG. 1(e) is a diagram illustrating Regnase-1 expression level in PBMC of PH patients (n=50) divided into two groups of high group (≥30%) (n=37) and low group (<30%) (n=13) in terms of right ventricular ejection fraction (RVEF) found in cardiac MRI. FIG. 1(f) is a diagram illustrating relationship between Regnase-1 expression level in PBMC and serum brain natriuretic peptide (BNP) level of PH patients (n=77). FIG. 1(g) is a diagram illustrating relationship between Regnase-1 expression level in PBMC and serum uric acid level of PH patients (n=77). FIGS. 1(h) and 1(i) are diagrams illustrating relationship between Regnase-1 expression level in PBMC and serum C-reactive protein (CRP) level of PH patients (n=77). FIG. 1(j) is a diagram illustrating Regnase-1 expression level in PBMC of PH patients (n=77) divided into two groups of patients (n=53) having been treated for PH and patients (n=24) not having been treated for pH. FIG. 1(k) is a diagram illustrating Regnase-1 expression level in PBMC of pH patients (n=53) having treated for PH, divided into two groups of high group (≥45 mmHG: n=15) and low group (<45 mmHG: n=38) in terms of mPAP.

FIG. 2(a) is a diagram illustrating Regnase-1 expression level in PBMC of PH patients (n=77) divided into subgroups. FIG. 2(b) is a diagram illustrating Regnase-1 expression levels in PBMC of CTD-PAH patients (n=22) divided into two groups of mild group (classes 1 and 2) (n=9) and severe group (classes 3 and 4) (n=13) according to WHO-FC. FIG. 2(c) is a diagram illustrating Regnase-1 expression level in PBMC of idiopathic PAH and heritable PAH (I/HPAH) patients (n=33) divided into two groups of mild group (classes 1 and 2) (n=20) and moderate-to-severe group (classes 3 and 4) (n=13) according to WHO-FC. FIG. 2(d) is a diagram illustrating relationship between Regnase-1 expression level in PBMC and mPAP of CTD-PAH patients (n=22). FIG. 2(e) is a diagram illustrating the relationship between Regnase-1 expression level in PBMC and six-minute walk distance (6MWD) of CTD-PAH patients (n=22). FIG. 2(f) is a diagram illustrating relationship between Regnase-1 expression level in PBMC and serum uric acid level of CTD-PAH patients (n=22). FIG. 2(g) is a diagram illustrating Regnase-1 expression levels in PBMC of CTD-PAH patients (n=22) divided into two groups of high group (≥0.3 mg/dl) (n=14) and low group (<0.3 mg/dl) (n=8) in terms of serum CRP level. FIG. 2(h) is a diagram illustrating relationship between Regnase-1 expression level in PBMC and mPAP of I/HPAH patients (n=33). FIG. 2(i) is a diagram illustrating relationship between Regnase-1 expression level in PBMC and six-minute walk distance (6MWD) of I/HPAH patients (n=33). FIG. 2(j) is a diagram illustrating relationship between Regnase-1 expression level in PBMC and serum uric acid level of I/HPAH patients (n=33). FIG. 2(k) is a diagram illustrating Regnase-1 expression level in PBMC of I/HPAH patients (n=33) divided into two groups of high group (≥0.3 mg/dl) (n=27) and low group (<0.3 mg/dl) (n=6) in terms of serum CRP level.

FIG. 3 illustrates measured results of expression levels of IL-6 mRNA and Regnase-1 mRNA in cell fractions recovered from bronchoalveolar lavage fluid of 8-week-old wild-type mice (C57BL/6), after being subjected to four-day hypoxia load (Hypoxia) to induce clinical condition of hypoxia-induced PH (n=6). In FIG. 3, “Normoxia” denotes results of control mice bred under normoxic condition (n=6).

FIG. 4 illustrates measured results of right ventricular systolic pressure (RVSP), Fulton coefficient (RV/LV+S), and expression levels of IL-6 mRNA and Regnase-1 mRNA in pulmonary tissue of 6-week-old SD rats, on day 4 after administration of monocrotaline (MCT) to induce clinical condition of PH (n=8). In FIG. 4, “Control” denotes results of control rats without administration of MCT (n=3).

FIG. 5(a) is a diagram illustrating Regnase-1 expression level in alveolar macrophage (AMΦ) and dendritic cell (cDC) isolated from CD11c-Cre⁺Zc3h12a^{fl/fl} mice and control mice, determined by QPCR. FIG. 5(b) contains observed tissue images of liver, duodenum, and kidney sections from a CD11c-Cre⁺Zc3h12a^{fl/fl} mouse and a control mouse, under hematoxylin-eosin staining.

FIG. 6(a) contains histological images of lung samples excised from a CD11c-Cre⁺Zc3h12a^{fl/fl} mouse and a control mouse, under hematoxylin-eosin staining. FIG. 6(b) illustrates histological images of lung samples excised from CD11c-Cre⁺Zc3h12a^{fl/fl} mouse and control mouse, under immunostaining of α-SMA and von Willebrand factor (vWF).

FIG. 6(c) is a diagram illustrating medial wall thickness of lungs from CD11c-Cre⁺Zc3h12a^{fl/fl} mice (n=4) and control mice (n=3). FIG. 6(d) contains Elastica van Gieson-stained images of pulmonary arteries from CD11c-Cre⁺Zc3h12a^{fl/fl} mice corresponded to grades 1 to 4 of the Heath-Edwards classification. FIG. 6(e) is a diagram illustrating occlusion rate of pulmonary arteries of the mice illustrated in FIG. 6(d). FIG. 6(f) is a diagram illustrating right ventricular systolic pressure (RVSP) of CD11c-Cre⁺Zc3h12a^{fl/fl} mice (n=9) and control mice (n=8). FIG. 6(g) is a diagram illustrating Fulton coefficient (RV/LV+S) of CD11c-Cre⁺Zc3h12a^{fl/fl} mice (9-week-old, n=10; and 3.5-month-old, n=10) and control mice (n=9). FIG. 6(h) contains Elastica van Gieson-stained images of pulmonary veins from CD11c-Cre⁺Zc3h12a^{fl/fl} mice and control mice. FIG. 6(i) is a diagram illustrating RVSP of Lysm^{Cre/+}Zc3h12a^{fl/fl} mice (n=12) and control mice (n=11). FIG. 6(j) is a diagram illustrating Fulton coefficient of Lysm^{Cre/+}Zc3h12a^{fl/fl} mice (13-week-old, n=14; and 5-month-old, n=4) and control mice (n=12). FIG. 6(k) contains histological images of lung samples excised from a Lysm^{Cre/+}Zc3h12a^{fl/fl} mouse and a control mouse, under hematoxylin-eosin staining. FIG. 6(l) contains histological images of lung samples excised from a Lysm^{Cre/+}Zc3h12a^{fl/fl} mouse and a control mouse, under EVG staining. FIG. 6(m) contains Elastica van Gieson-stained images of pulmonary arteries from a Lysm^{Cre/+}Zc3h12a^{fl/fl} mouse and a control mouse.

FIG. 7(a) contains diagrams illustrating the individual cell counts of alveolar macrophage (AMΦ), CD11b⁺cDC2, CD103⁺cDC1, interstitial macrophage (IM), eosinophil, neutrophil, T cell, and B cell, contained in lung samples of CD11c-Cre⁺Zc3h12a^{fl/fl} mice (cko) (n=4) and control mice (n=4). FIG. 7(b) contains diagrams illustrating expression levels of CD40 and CD80 in CD11b⁺cDC2 and CD103⁺cDC1 from CD11c-Cre⁺Zc3h12a^{fl/fl} mice and control mice. FIG. 7(c) contains diagrams illustrating expression levels of Il1b and Il6 in cDCs from CD11c-Cre⁺Zc3h12a^{fl/fl} mice (cko) (n=3) and control mice (n=3). FIG. 7(d) contains diagrams illustrating expression levels of Il1b and Il6 in macrophages from CD11c-Cre⁺Zc3h12a^{fl/fl} mice (cko) (n=3) and control mice (n=3).

FIG. 8(a) contains diagrams illustrating expression levels of Regnase-1 in alveolar macrophages (AM) and interstitial macrophages (IM) obtained from lung samples of Lysm^{Cre/+}Zc3h12a^{fl/fl} mice (n=3) and control mice (n=3). FIG. 8(b) contains diagrams illustrating expression levels of Il6, Il1b, Arg1, Retnla, and Il4R in alveolar macrophages (AM) and interstitial macrophages (IM) from Lysm^{Cre/+}Zc3h12a^{fl/fl} mice (n=3) and control mice (n=3).

FIG. 9(a) contains histological images of lung samples from a CD11c-Cre⁺Zc3h12a^{fl/fl}Rag2^{-/-} mouse, under EVG staining. FIG. 9(b) is a drawing illustrating occlusion rate of pulmonary arteries from CD11c-Cre⁺Zc3h12a^{fl/fl}Rag2^{-/-} mice. FIG. 9(c) contains histological images of lung samples from Lysm^{Cre/+}Zc3h12a^{fl/fl}Rag2^{-/-} mouse, under EVG staining. FIG. 9(d) is a drawing illustrating occlusion rate of pulmonary arteries from CD11c-Lysm^{Cre/+}Zc3h12a^{flfl}Rag2^{-/-} mice. FIG. 9(e) is a diagram illustrating a protocol of experiment in which a CD11c-Cre⁺Zc3h12a^{fl/fl}Rag2^{-/-} mouse is treated with clodronic acid. FIG. 9(f) contains diagrams illustrating the individual counts of alveolar macrophages (AM) and interstitial macrophages (IM) from CD11c-Cre⁺Zc3h12a^{fl/fl}Rag2^{-/-} mice (CL; n=6, PBS; 4) and control mice (CL; n=4, PBS; 7) with intratracheal administration of clodronic acid (CL) or PBS. FIG. 9(g) contains histological images of lungs from a CD11c-Cre⁺Zc3h12a^{fl/fl}Rag2^{-/-} mouse and a control mouse, with intratracheal administration of clodronic acid (CL) or PBS, under EVG staining. FIG. 9(h) is a diagram illustrating medial wall thickness of lungs from CD11c-Cre⁺Zc3h12a^{fl/fl}Rag2^{-/-} mice (n=7) and control mice (n=4) with intratracheal administration of clodronic acid (CL) or PBS. FIG. 9(i) is a diagram illustrating occlusion rate of pulmonary arteries from CD11c-Cre⁺Zc3h12a^{fl/fl}Rag2^{-/-} mice and (n=7) and control mice (n=4) with intratracheal administration of clodronic acid (CL) or PBS. FIG. 9(j) is a diagram illustrating RVSP of CD11c-Cre⁺Zc3h12a^{fl/fl} mice (n=9) and control mice (n=7) bred under hypoxia load, and CD11c-Cre⁺Zc3h12a^{fl/fl} mice (n=6) and control mice (n=5) bred under nomoxia. FIG. 9(k) is a diagram illustrating Fulton coefficient of CD11c-Cre⁺Zc3h12a^{fl/fl} mice (n=9) and control mice (n=7), and CD11c-Cre⁺Zc3h12a^{f/fl} mice (n=6) and control mouse (n=5) bred under nomoxia.

FIG. 10-1(a) is a diagram illustrating a protocol for transcriptome analysis of pulmonary artery and alveolar macrophage from a CD11c-Cre⁺Zc3h12a^{fl/fl} mouse. FIG. 10-1(b) is a chart illustrating gene group in which genes increased in the pulmonary artery of CD11c-Cre⁺Zc3h12a^{fl/fl} mice were highly enriched. FIG. 10-1(c) is a diagram illustrating results of the gene set enrichment analysis (GSEA) (Kyoto Encyclopedia of Genes and Genomes) for gene sets related to cell cycle, among genes whose expression increased in pulmonary artery of CD11c-Cre⁺Zc3h12a^{fl/fl} mice. FIG. 10-1(d) is a diagram illustrating results of the KEGG pathway analysis for genes upregulated in pulmonary artery of CD11c-Cre⁺Zc3h12a^{fl/fl} mice. FIG. 10-1(e) is a diagram illustrating a volcano plot of transcriptome analysis results with use of alveolar macrophages from CD11c-Cre⁺Zc3h12a^{fl/fl} mice and control mice. FIG. 10-1(f) is a diagram illustrating a heat map of expression levels of angiogenic cytokines in alveolar macrophages from CD11c-Cre⁺Zc3h12a^{fl/fl} mice and control mice. FIG. 10-1(g) is a diagram illustrating a heat map of expression levels of M1 and M2 macrophage signature genes in alveolar macrophages from CD11c-Cre⁺Zc3h12a^{fl/fl} mice and control mice. FIG. 10-2(h) is a ligand-target matrix illustrating regulatory potential between Regnase-1 deficient alveolar macrophage ligand and target genes in pulmonary artery. FIG. 10-3(i) is a ligand-target matrix illustrating regulatory potential between Regnase-1 deficient alveolar macrophage ligand and target genes in pulmonary artery.

FIG. 11 contains diagrams illustrating expression levels of Il1b, Il6, Il10, Arg1, Retnla, Ym1, Il4ra, Ir4f, Ccl24, Pdgfa, Pdgfb, Vegfa, Fgf2, Hgf, Igf1, Plau, and Thbs1 in alveolar macrophages from CD11c-Cre⁺Zc3h12a^{fl/fl} mice (n=3) and control mice (n=3).

FIG. 12(a) contains diagrams illustrating results (n=3) of luciferase reporter assay conducted by transfecting HEK293 cells with luciferase reporter plasmid pGL3 that contains 3′UTR of gene, and expression plasmid of Regnase-1 (WT), Regnase-1 mutant (D141N), or empty (control). FIG. 12(b) is a diagram illustrating a protocol of therapeutic experiment on a PAH model mouse with use of anti-IL-6 receptor-neutralizing antibody (MR16-1). FIG. 12(c) is a diagram illustrating RVSP of CD11c-Cre⁺Zc3h12a^{fl/fl} mice with administration of MR16-1 or control antibody (IgG). FIG. 12(d) is a diagram illustrating Fulton coefficient of CD11c-Cre⁺Zc3h12a^{fl/fl} mice with administration of MR16-1 or control antibody (IgG). FIG. 12(e) contains histological images of lung samples excised from CD11c-Cre⁺Zc3h12a^{fl/fl} mice with administration of MR16-1 or control antibody (IgG), under hematoxylin-eosin staining. FIG. 12(f) is a diagram illustrating a protocol of therapeutic experiment on a PAH model mouse with use of PDGF receptor inhibitor (imatinib) and IL-1 receptor inhibitor (anakinra). FIG. 12(g) is a diagram illustrating RVSP of CD11c-Cre⁺Zc3h12a^{fl/fl} mice with administration of imatinib or anakinra. FIG. 12(h) is a diagram illustrating Fulton coefficient of CD11c-Cre⁺Zc3h12a^{fl/fl} mice with administration of imatinib or anakinra.

FIG. 13(a) is a diagram illustrating a protocol of PAH therapeutic experiment with use of Regnase-1 morpholino oligo (Reg1 MO). FIG. 13(b) is a diagram illustrating RVSP of mice with administration of Reg1 MO or control oligo, and bred under hypoxia load. FIG. 13(c) is a diagram illustrating Fulton coefficient of mice with administration of Reg1 MO or control oligo, and bred under hypoxia load. FIG. 13(d) contains histological images of lung samples excised from mice with administration of Reg1 MO or control oligo, and bread under hypoxia load. FIG. 13(e) is a diagram illustrating medial wall thickness coefficient of lungs from mice with administration of Reg1MO or control oligo, and bred under hypoxia load.

### DESCRIPTION OF EMBODIMENTS

### 1. Terms

Unless specifically defined otherwise, all terms used herein have meanings as commonly understood by those skilled typically in the art of medicine, pharmacy, molecular biology, microbiology, organic and chemistry. For any term defined herein, but not used in the meanings as commonly understood, the description herein prevails.

Pulmonary hypertension (PH) is a progressive disease group with poor prognosis, which causes cardiac and pulmonary dysfunctions due to elevated pulmonary artery pressure, and is classified by the Nice Classification into Group 1: pulmonary arterial hypertension (PAH); Group 1′: pulmonary atresia (pulmonary veno-occlusive disease, PVOD) and/or pulmonary capillary hemangiomatosis (PCH); Group 1″: persistent pulmonary hypertension of the newborn; Group 2: pulmonary hypertension due to left heart disease; Group 3: pulmonary hypertension due to pulmonary disease and/or hypoxemia; Group 4: chronic thromboembolic pulmonary hypertension (CTEPH), and Group 5: pulmonary hypertension associated with multifactorial mechanism with unknown details.

Pulmonary arterial hypertension (PAH) is further subclassified into idiopathic PAH (IPAH), heritable PAH (HPAH), drug- or toxin-induced PAH, and PAH associated with various diseases (associated PAH). PAH associated with various diseases includes those ascribed to connective tissue disease (CTD), human immunodeficiency virus (HIV) infection, portal hypertension, congenital heart disease, and schistosomiasis.

### 2. Invention with Use of PH Marker

### 2-1. Test Method

One embodiment of the present disclosure relates to a method for testing presence/absence, severity, or prognosis of pulmonary hypertension, the test method including: measuring Regnase-1 gene expression level or Regnase-1 level in a sample isolated from a subject.

In the present disclosure, the “subject” is a human or non-human animal for which the presence/absence of contract with PH, severity of PH, or prognosis of PH is to be determined. Specific examples of the non-human animal include non-human mammals such as primate, rat, mouse, gerbil, guinea pig, hamster, ferret, rabbit, cow, horse, pig, goat, dog, and cat. The test method of the present disclosure is suitable for a test for human, so that the subject is preferably human.

The “sample isolated from a subject” is a biological sample isolated from the aforementioned subject. The sample is preferably blood of the subject, or a sample prepared from the blood. In particular, the test method of the present disclosure enables precise examination while using Regnase-1 gene expression level of Regnase-1 level in peripheral blood mononuclear cell as an index, so that preferred is peripheral blood, sample that contains peripheral blood mononuclear cell prepared from peripheral blood, or isolated peripheral blood mononuclear cell.

The test method of the present disclosure may measure either one of, or both of the Regnase-1 gene expression level and Regnase-1 level (the amount of Regnase-1 protein).

The Regnase-1 gene expression level in the sample may be measured by any of known methods including Northern blotting, RT-PCR, real time RT-PCR, RNA-Seq analysis, DNA microarray method (method with use of DNA chip), dot blotting, and RNase protection assay. Measured value of the Regnase-1 gene expression level may be a relative expression level of Regnase-1 gene, with reference to gene expression level(s) of one or more endogenous controls in the sample. The endogenous control usable herein may be a housekeeping gene.

The Regnase-1 in the sample may be measured typically by immunoassay with use of an antibody that specifically recognizes and binds to Regnase-1. The antibody may be prepared by any of known methods. The immunoassay is exemplified by a method with use of a solid phase carrier having an antibody that specifically binds to Regnase-1 immobilized thereon, flow cytometry, and Western blotting. The method with use of a solid phase carrier is exemplified by enzyme-linked immunosorbent assay (ELISA) with use of immobilized microtiter plate, and aggregation method (immunoprecipitation method) with use of immobilized particle. The Regnase-1 may alternatively be measured by a method typically based on multiple reaction monitoring (MRM) by liquid chromatography-mass spectrometry (LC-MS/MS), which is an antibody-free protein mass spectrometric technique. Also these detection methods may be conducted according to common processes.

PH aimed at by the test method of the present disclosure may be any of those in Group 1, Group 1′, Group 1″, Group 2, Group 3, Group 4, and Group 5 of the Nice Classification. Preferred PH to be tested is exemplified by Group 1 (PAH). In particular, decrease in the Regnase-1 gene expression level and Regnase-1 level is likely to reflect clinical condition of idiopathic or hereditary PAH (I/HPAH) and PAH due to connective tissue disease (CTD-PAH), so that preferred PH aimed at by the test method in one embodiment of the present disclosure is exemplified by I/HPAH and CTD-PAH.

In the testing of presence/absence of PH by the test method of the present disclosure, judgement will be made on that the lower the Regnase-1 gene expression level or the Regnase-1 level, the more likely the subject contract with PH. In the testing of severity of PH by the test method of the present disclosure, judgement will be made on that the lower the Regnase-1 gene expression level or the Regnase-1 level, the severer the PH of the subject. In the testing of prognosis of PH by the test method of the present disclosure, judgement will be made on that the lower the Regnase-1 gene expression level or the Regnase-1 level, the poorer the prognosis of PH of the subject.

In the test method of the present disclosure, whether the Regnase-1 gene expression level or the Regnase-1 level is low or not may be determined, by comparing the Regnase-1 gene expression level or the Regnase-1 level in the sample, with a reference value. The “reference value” herein is a Regnase-1 gene expression level or a Regnase-1 level in the sample obtained from a person whose presence/absence, severity, or prognosis of PH, for example, has been known.

In an exemplary case of testing presence/absence of PH, an average value of the Regnase-1 gene expression levels or the Regnase-1 levels in the samples from a plurality of healthy subjects is determined in advance, and with use of the average value as a reference value, the subject may be judged to possibly contract with PH, if the Regnase-1 gene expression level or the Regnase-1 level in the sample from the subject is lower than the reference value. In another exemplary case of testing presence/absence of PH, an average value of the Regnase-1 gene expression levels or the Regnase-1 levels in the samples from a plurality of PH patients is determined in advance, and with use of the average value as a reference value, the subject may be judged to possibly contract with PH, if the Regnase-1 gene expression level or the Regnase-1 level in the sample from the subject is equivalent to or below the reference value.

In an exemplary case of testing severity of PH, an average value of the Regnase-1 gene expression levels or the Regnase-1 levels in the samples from PH patients with known grades of severity, is determined in advance for each grade of severity, and with use of the average values as reference values, the severity of PH may be judged by determining to which reference value, the Regnase-1 gene expression level or the Regnase-1 level of the sample of the subject is closest.

In an exemplary case of testing prognosis of PH, an average value of the Regnase-1 gene expression levels or the Regnase-1 levels in the samples from PH patients with known prognosis in relation to presence/absence of events (including heart failure, death, lung transplant), is determined in advance according to presence/absence of each event, and with use of the average values as reference values, the prognosis of PH may be predicted by determining to which reference value, the Regnase-1 gene expression level or the Regnase-1 level of the sample of the subject is closest.

### 2-2. Test Kit

Another embodiment of the present disclosure relates to a test kit for testing presence/absence, severity, or prognosis of PH, wherein the test kit contains a reagent for measuring the Regnase-1 gene expression level or the Regnase-1 level. The test kit of the present disclosure is a test kit used for carrying out the aforementioned test method. The contents described in section “1.2-1. Test Method” are also incorporated by reference into the test kit of the present disclosure.

Examples of the reagent for measuring the Regnase-1 gene expression level include primer pair for amplifying nucleic acid that contains Regnase-1 gene (for example, mRNA, cDNA derived from mRNA), and probe that hybridizes with the nucleic acid.

Meanwhile, examples of the reagent for measuring the Regnase-1 level include antibody that specifically binds to Regnase-1. The antibody may be either polyclonal antibody or monoclonal antibody. The antibody may also be antibody fragment, as long as it can specifically bind to Regnase-1. Examples of the antibody fragment include Fab fragment, F(ab′)2 fragment, and single-chain antibody (scFv). The antibody may be provided in a form immobilized on a solid phase carrier, such as microtiter plate or particle.

The test kit of the present disclosure may further contain buffer solution for dilution or reaction, with component necessary for the measurement contained therein, washing liquid, chromogenic reagent, reaction container, and the like.

### 3. PAH Pathological Model Animal and Method with Use of Same

### 3-1. PAH Pathological Model Animal

Yet another embodiment of the present disclosure relates to a pathological model animal for PAH, consisting of a non-human animal with Regnase-1 deficiency in at least one immune cell. The PAH pathological model animal of the present disclosure will be explained below.

Species of the PAH pathological model animal of the present disclosure is exemplified by, but not specifically limited to, non-human mammals such as primate, rat, mouse, gerbil, guinea pig, hamster, ferret, rabbit, cow, horse, pig, goat, dog, cat, and monkey. Among these non-human mammals, preferred examples include mouse, rat, guinea pig, and rabbit.

In the PAH pathological model animal of the present disclosure, type of immune cell to be made deficient in regase-1 is exemplified by, but not particularly limited to, macrophages such as alveolar macrophage; dendritic cell; granulocytes such as neutrophil, eosinophil, and basophil; and lymphocytes such as T cell and B cell. One preferred embodiment of the PAH pathological model animal of the present disclosure is exemplified by non-human animal with Regnase-1 deficiency in at least in alveolar macrophage. The non-human animal with Regnase-1 deficiency in at least in alveolar macrophage may further have Regnase-1 deficiency in at least one or more of macrophage, lymphocyte, and granulocyte. One embodiment of the PAH pathological model animal of the present disclosure is exemplified by non-human animal deficient in Regnase-1 in at least in alveolar macrophage and dendritic cell. Another embodiment of the PAH pathological model animal of the present disclosure is exemplified by non-human animal with Regnase-1 deficiency in at least in alveolar macrophage and myeloid dendritic cell.

The non-human animal with Regnase-1 deficiency in an immune cell-specific manner may be created by the conditional knockout methods. The conditional knockout method may be conducted by any of known method typically with use of a Cre/loxP system. For example, a non-human animal with Regnase-1 deficiency in alveolar macrophage may be created, by creating an animal (flox animal) having a gene locus in which a gene (ZC3H12 A) region that encodes Regnase-1 is sandwiched by Cre recombinase target sequences loxP, and then by crossing the animal with an animal having the Cre recombinase already expressed in alveolar macrophage.

For example, a CD11c-Cre mouse, induced by a CD11c gene promoter and having Cre recombinase which is a DNA recombinant enzyme introduced therein, has been known to delete a target gene specifically in alveolar macrophage and dendritic cell, as a result of crossing with a flox animal. Therefore, crossing of the CD11c-Cre mouse with a mouse having a flox site introduced into the Zc3h12a gene, can create a mouse with Regnase-1 deficiency specifically in alveolar macrophage and dendritic cell.

In another example, a LysM-Cre mouse, induced by a lysozyme gene promoter and having Cre recombinase which is a DNA recombinant enzyme introduced therein, has been known to delete a target gene specifically in myeloid cell including alveolar macrophage, as a result of crossing with a flox animal. Therefore, crossing of the LysM-Cre mouse, with a mouse having a flox site introduced into the Zc3h12a gene, can create a mouse with Regnase-1 deficiency specifically in myeloid immune cell including alveolar macrophage.

The prior creation of PAH pathological model animal has needed hypoxia load or exposure to a chemical substance such as monocrotaline or Sugen 5416. In contrast, the non-human animal with Regnase-1 deficiency in at least one immune cell, particularly in alveolar macrophage, can spontaneously develop PAH and exhibits pathological condition of PAH, even bred in a normal breeding environment. A breeding period over which the PAH pathological model animal of the present disclosure spontaneously develops PAH is usually approx. 6 to 7 weeks, although variable typically depending on the species and strain of the animal, and the type of the immune cell causing Regnase-1 deficiency. Presence of clinical condition of PAH may be confirmed typically by hemodynamic measurement or morphological analysis of pulmonary tissue and cardiac tissue, with reference to right ventricular systolic pressure (RVSP), Fulton coefficient (weight ratio of right ventricle/(left ventricle+septum)) as an index of the degree of right ventricular hypertrophy, or pulmonary vascular remodeling (stenosis or occlusion).

The PAH pathological model animal of the present disclosure is usable as a PAH pathological model for any of I/HPAH, CTD-PAH, CHD-PAH, portal vein pulmonary hypertension (PoPH), and drug-induced PAH. One embodiment of the PAH pathological model animal is suitably used as an I/HPAH pathological model or a CTD-PAH pathological model, for its easy reflectance of clinical conditions of I/HPAH and CTD-PAH.

The PAH pathological model animal of the present disclosure may be usable not only for screening of a prophylactic or therapeutic drug for PAH, or for assessing medicinal effect of a prophylactic or therapeutic drug for PAH as described later, but also as a research sample for elucidating pathology of PAH.

3-2. Method for Screening Prophylactic or Therapeutic Drug for PAH, with Use of PAH Pathological Model Animal

Yet another embodiment of the present invention relates to a method for screening a candidate substance that is applicable to a prophylactic or therapeutic drug for pulmonary arterial hypertension from among test substances, the screening method including first to third steps below:
a first step of administrating a test substance to a non-human animal with Regnase-1 deficiency in at least one immune cell;
a second step of examining a clinical condition of pulmonary arterial hypertension of the non-human animal with administration of the test substance; and
a third step of selecting the test substance as a candidate substance that can be used as a prophylactic or therapeutic drug for pulmonary arterial hypertension, if the clinical condition of pulmonary arterial hypertension in the non-human animal with administration of the test substance is found to improve as compared with that in the non-human animal without administration of the test substance.

### [First Step]

In the first step, the test substance is administered to a non-human animal with Regnase-1 deficiency in at least one immune cell.

In the screening method of the present disclosure, the test substance is a substance to be determined whether or not it can demonstrate a prophylactic and/or therapeutic effect. Specific examples of the test substance include synthetic compound, nucleic acid (such as antisense nucleic acid, cDNA, siRNA, for example), peptide, protein, organic compound, inorganic compound, cell extract, cell culture supernatant, plant extract, culture product, and mixtures thereof.

The “non-human animal with Regnase-1 deficiency in at least one immune cell” used in the first step is as described in the aforementioned section “3-1. PAH Pathological Model Animal”.

In the first step, the test substance may be administered to the non-human animal at any time not particularly limited, either before or after the non-human animal develops clinical condition of PAH. The administration may continue over a pre-onset period to a post-onset period of PAH. With the test substance administered, in the first step, to the non-human animal in the pre-onset period of PAH, this makes it possible to screen a candidate substance having an effect of delaying the onset of PAH or an effect of suppressing the onset of PAH. Meanwhile, with the test substance administered, in the second step, to the non-human animal in the pre-onset period of PAH, this makes it possible to screen a candidate substance having an effect of improving clinical condition of PAH.

The number of times the test substance is administered to the non-human animal in the first step is not particularly limited, so that the administration of the test substance may take place only once, or twice or more at regular intervals.

Method for administering the test substance to the non-human animal in the first step is not particularly limited, and may be any of oral administration, intravascular (intraarterial or intravenous) injection, pulmonary administration (inhalation), continuous infusion, subcutaneous administration, intramuscular administration, enteral administration, intraperitoneal administration, topical administration, and so forth.

### [Second Step]

In the second step, the clinical condition of PAH in the non-human animal with administration of the test substance is examined. In the second step, the clinical condition of PAH may be examined at any time not particularly limited, and may only be at a point in time the effect of the test substance is sufficiently demonstrated, which may be approx. day 14 from last administration of the test substance, or may be about day 14 to day 56, or about day 28 to day 56.

In the second step, the clinical condition of PAH may be confirmed typically by hemodynamic measurement or morphological analysis of pulmonary tissue and cardiac tissue, with reference to right ventricular systolic pressure (RVSP), weight ratio of right ventricle/left ventricle (Fulton coefficient), or pulmonary vascular remodeling.

### [Third Step]

In the third step, the test substance is selected as a candidate substance that can be used as a prophylactic or therapeutic drug for PAH, if the clinical condition of PAH in the non-human animal with administration of the test substance is found to improve as compared with that in the non-human animal without administration of the test substance.

The candidate substance selected in the third step can be a candidate for a prophylactic or therapeutic drug for any types of PAH including I/HPAH, CTD-PAH, CHD-PAH, PoPH, and drug-induced PAH. The candidate substance selected in the third step is preferred as a candidate for a prophylactic or therapeutic drug for I/HPAH or CTD-PAH, for its easy reflectance of clinical conditions of I/HPAH and CTD-PAH in the non-human animal.

The candidate substance selected in the third step may further be subjected to a test typically for assessing safety or clinical trial, so as to assess clinical efficacy as a prophylactic or therapeutic drug for PAH.

3-3. Method for Assessing Drug Efficacy for PAH, with Use of PAH Pathological Model Animal

Yet another embodiment of the present disclosure relates to a method for assessing medicinal effect of a test substance on PAH, the medicinal effect assessing method including step I and step II below:
step I of administering a test substance to a non-human animal with Regnase-1 deficiency in at least one immune cell; and
step II of examining a clinical condition of PAH in the non-human animal with administration of the test substance.

In the medicinal effect assessing method of the present disclosure, PAH to be assessed for the medicinal effect may be any of I/HPAH, CTD-PAH, CHD-PAH, PoPH, and drug-induced PAH. The method is suitable for assessing the medicinal effect on I/HPAH or CTD-PAH, for its easy reflectance of clinical conditions of I/HPAH and CTD-PAH in the non-human animal.

### [Step I]

In step I, the test substance is administered to a non-human animal with Regnase-1 deficiency in at least one immune cell.

In the method for assessing medicinal effect of the present disclosure, the test substance is a substance to be determined whether or not it can demonstrate a medicinal effect (prophylactic and/or therapeutic effect) for PAH. The test substance may be a substance proven or suggested to have medicinal effect as a prophylactic or therapeutic drug for PAH, or may be a substance not proven to have prophylactic and/or therapeutic effect for PAH. Specific examples of the test substance include synthetic compound, nucleic acid (such as antisense nucleic acid, cDNA, siRNA, for example), peptide, protein, organic compound, inorganic compound, cell extract, cell culture supernatant, plant extract, culture product, and mixtures thereof.

The “non-human animal with Regnase-1 deficiency in at least one immune cell” used in step I is as described in the aforementioned section “3-1. PAH Pathological Model Animal”.

The timing, the number of times, the method and the like of administration of the test substance to the non-human animal in step I are the same as those in the first step of the “3-2. Method for Screening Prophylactic or Therapeutic Drug for PAH with Use of PAH Pathological Model Animal”.

### [Step II]

In step II, the clinical condition of PAH in the non-human animal with administration of the test substance is examined. The timing or method for examining the clinical condition of PAH in step II are the same as those in the second step of the “3-2. Method for Screening Prophylactic or Therapeutic Drug for PAH with Use of PAH Pathological Model Animal”. In step II, judgement will be made on that the higher the degree of improvement in the clinical condition of PAH, the higher the medicinal effect of the test substance on PAH.

### 4. Prophylactic or Therapeutic Drug for PH

Yet another embodiment of the present disclosure relates to a prophylactic or therapeutic drug for PH, containing a substance that disrupts a stem-loop structure in the 3′UTR of Regnase-1 mRNA. The prophylactic or therapeutic drug for PH of the present disclosure will be explained below.

[Substance that Decomposes Stem-Loop Structure in 3′UTR of Regnase-1 mRNA]

Regnase-1 has been known to recognize two stem-loop structures present in the 3′UTR of Regnase-1 mRNA, and to disrupt Regnase-1 mRNA (WO2019/182055). The prophylactic or therapeutic drug for PH of the present disclosure can suppress decrease in the Regnase-1 expression level, by decomposing at least one of the two stem-loop structures in the 3′UTR of Regnase-1 mRNA, thereby improving the clinical condition of PH.

In the prophylactic or therapeutic drug for PH of the present disclosure, Regnase-1 that disrupts the stem-loop structure may only be derived from a species to which the prophylactic or therapeutic drug for PH is administered. In an exemplary case where the prophylactic or therapeutic drug for PH is administered to human, usable is a substance that disrupts the stem-loop structure in the 3′UTR of human Regnase-1 mRNA. Representative amino acid sequences of Regnase-1 are registered under GenBank Accession Numbers of NP_001310479.1 (human, SEQ ID NO: 3) and NP_694799.1 (mouse, SEQ ID NO: 4).

The “3′UTR of Regnase-1 mRNA” is a 3′ untranslated region in Regnase-1 mRNA. The base sequence of the 3′UTR of Regnase-1 mRNA is exemplified by a base sequence that contains at least a sequence represented by SEQ ID NO: 5 (human) or SEQ ID NO: 6 (mouse). For example, the 3′UTR of Regnase-1 mRNA contains a base sequence represented by SEQ ID NO: 1 (human) or SEQ ID NO: 2 (mouse).

The “stem-loop structure” is a structure whose stem is formed by binding of complementary sequences that resides apart at two regions in a molecule of single-stranded nucleic acid, while leaving a loop between the two regions. The 3′UTR of human Regnase-1 mRNA has a first stem-loop structure formed in a region that corresponds to positions 231 to 245 of SEQ ID NO: 1, and/or, a second stem-loop structure formed in a region that corresponds to positions 424 to 442 of SEQ ID NO: 1. Meanwhile, the 3′UTR of mouse Regnase-1 mRNA has a first stem-loop structure formed in a region that corresponds to positions 196 to 210 of SEQ ID NO: 2, and/or, a second stem-loop structure formed in a region that corresponds to positions 378 to 392 of SEQ ID NO: 2.

The “substance that disrupts the stem-loop structure” is a substance that inhibits complementary binding in the stem-loop structure. Substances that disrupt the stem-loop structure in the 3′UTR of mouse Regnase-1 mRNA have been known, as described in WO 2019/182055. The substance that disrupts the stem-loop structure may only be a substance that inhibits complementary binding in at least one pair, at least two pairs, or at least three pairs of nucleotide in the stem-loop structure. Such substance is exemplified by an oligonucleic acid that binds to a base sequence that forms the stem-loop structure, and a substance for modifying a base sequence that forms the stem-loop structure by a genome editing technique.

In one embodiment of the prophylactic or therapeutic drug for PH of the present disclosure, the substance that disrupts the stem-loop structure in the 3′UTR of Regnase-1 mRNA is an oligonucleic acid that hybridizes to at least a part of a base sequence in a region that forms the stem-loop structure. The oligonucleic acid can inhibit complementary binding in the stem-loop structure, by hybridizing itself to at least a part of a region that forms the stem-loop structure in the 3′UTR of Regnase-1 mRNA, which is typically at least one, at least two, or at least three nucleotides that form the stem portion of the stem-loop structure. For example, the oligonucleic acid contains a sequence complementary to a sequence that contains at least two or three, and typically three consecutive nucleotides that form the stem portion of the stem-loop structure. Such consecutive nucleotides can be positioned adjacent to the loop of the stem-loop structure, and may correspond, for example, to positions 233 to 235, positions 241 to 243, positions 426 to 428, and positions 438 to 440 in SEQ ID NO: 1. The oligonucleic acid itself preferably does not form a stem-loop structure, a hairpin structure, or a multimer such as dimer. The oligonucleic acid is typically a single-stranded nucleic acid composed of 10 to 30 bases, 15 to 27 bases, 18 to 25 bases, or 20 to 23 bases.

In one embodiment, the oligonucleic acid capable of decomposing the stem-loop structure in the 3′UTR of human Regnase-1 mRNA is at least either oligonucleic acid (a-1) or (a-2) below:
(a-1) an oligonucleic acid 10 to 30 bases in length that hybridizes to a sequence having 10 to 30 consecutive bases including positions 233 to 235 in SEQ ID NO: 1, within a base sequence from positions 206 to 242 in SEQ ID NO: 1;
(a-2) an oligonucleic acid 10 to 30 bases in length that hybridizes to a sequence having 10 to 30 consecutive bases including positions 241 to 243 in SEQ ID NO: 1, within a base sequence from positions 234 to 270 in SEQ ID NO: 1;

In one embodiment, the oligonucleic acid (a-1) has a sequence identity to SEQ ID NO: 7 (5′-AATGTGTATCAACAGGGTGATCG-3′) of at least approx. 60%, approx. 70%, approx. 80%, approx. 85%, approx. 90%, approx. 95%, approx. 96%, approx. 97%, approx. 98%, or approx. 99% or larger. In another embodiment, the oligonucleic acid (a-1) is composed of a base sequence of SEQ ID NO: 7, in which one or several bases, typically two or three bases, are deleted, substituted, added or inserted. In one embodiment, the oligonucleic acid (a-1) is composed of the sequence of SEQ ID NO: 7, or contains the base sequence of SEQ ID NO: 7.

In the present disclosure, the sequence identity means a degree of identity of base sequence, and is determined by comparing two sequences optimally aligned (so as to maximize the nucleotide matching) over the regions of the sequences to be compared. Numerical value (%) of sequence identity is calculated by determining identical bases present in both sequences, determining the number of matched sites, then dividing the number of matched sites by the total number of bases in the sequence region to be compared, and multiplying the obtained quotient by 100. Algorithms for obtaining optimal alignment and sequence identity include various algorithms commonly available to those skilled in the art (such as BLAST algorithm, and FASTA algorithm, for example). The sequence identity may be determined, typically with use of sequence analysis software such as BLAST or FASTA.

In one embodiment, the oligonucleic acid (a-2) has a sequence identity to SEQ ID NO: 8 (5′-CTTAAACTACAGAGATACAATGT-3′) of at least approx. 60%, approx. 70%, approx. 80%, approx. 85%, approx. 90%, approx. 95%, approx. 96%, approx. 97%, approx. 98%, or approx. 99% or larger. In another embodiment, the oligonucleic acid (a-2) is composed of a base sequence of SEQ ID NO: 8, in which one or several bases, typically two or three bases, are deleted, substituted, added or inserted. In one mode and in one embodiment, the oligonucleic acid (a-2) is composed of the sequence of SEQ ID NO: 8, or contains the base sequence of SEQ ID NO: 8.

In another embodiment, the oligonucleic acid that can disrupt the stem-loop structure in the 3′UTR of human Regnase-1 mRNA is at least either oligonucleic acid (b-1) or (b-2) below:
(b-1) an oligonucleic acid 10 to 30 bases in length that hybridizes to a sequence having 10 to 30 consecutive bases including positions 426 to 428 in SEQ ID NO: 1, within a base sequence from positions 399 to 439 in SEQ ID NO: 1; and
(b-2) an oligonucleic acid 10 to 30 bases in length that hybridizes to a sequence having 10 to 30 consecutive bases including positions 438 to 440 in SEQ ID NO: 1, within a base sequence from positions 427 to 467 in SEQ ID NO: 1.

In one embodiment, the oligonucleic acid (b-1) has a sequence identity to SEQ ID NO: 9 (5′-ACGGTGCCCAACTAGCCAG-3′) of at least approx. 60%, approx. 70%, approx. 80%, approx. 85%, approx. 90%, approx. 95%, approx. 96%, approx. 97%, approx. 98%, or approx. 99% or larger. In another embodiment, the oligonucleic acid (b-1) is composed of a base sequence of SEQ ID NO: 9, in which one or several bases, typically two or three bases, are deleted, substituted, added or inserted. In one embodiment, the oligonucleic acid (b-1) is composed of the sequence of SEQ ID NO: 9, or contains the base sequence of SEQ ID NO: 9.

In one embodiment, the oligonucleic acid (b-2) has a sequence identity to SEQ ID NO: 10 (5′-GGCCCTTGGAGGGCAGGCA-3′) of at least approx. 60%, approx. 70%, approx. 80%, approx. 85%, approx. 90%, approx. 95%, approx. 96%, approx. 97%, approx. 98%, or approx. 99% or larger. In another embodiment, the oligonucleic acid (b-2) is composed of a base sequence of SEQ ID NO: 10, in which one or several bases, typically two or three bases, are deleted, substituted, added or inserted. In one embodiment, the oligonucleic acid (b-2) is composed of the sequence of SEQ ID NO: 10, or contains the base sequence of SEQ ID NO: 10.

In one embodiment, the oligonucleic acid that can disrupt the stem-loop structure in the 3′UTR of mouse Regnase-1 mRNA is at least either oligonucleic acid (a-1′) or (a-2′) below:
(a-1′) an oligonucleic acid 10 to 30 bases in length that hybridizes to a sequence having 10 to 30 consecutive bases including positions 198 to 200 in SEQ ID NO: 2, within a base sequence from positions 171 to 207 in SEQ ID NO: 2; and
(a-2′) an oligonucleic acid 10 to 30 bases in length that hybridizes to a sequence having 10 to 30 consecutive bases including positions 206 to 208 in SEQ ID NO: 2, within a base sequence from positions 199 to 235 in SEQ ID NO: 2.

In one embodiment, the oligonucleic acid (a-1′) has a sequence identity to SEQ ID NO: 11 (5′-aatgtgtatcaacagggtgatca-3′) of at least approx. 60%, approx. 70%, approx. 80%, approx. 85%, approx. 90%, approx. 95%, approx. 96%, approx. 97%, approx. 98%, or approx. 99% or larger. In another embodiment, the oligonucleic acid (a-1′) is composed of a base sequence of SEQ ID NO: 11, in which one or several bases, typically two or three bases, are deleted, substituted, added or inserted. In one embodiment, the oligonucleic acid (a-1′) is composed of the sequence of SEQ ID NO: 11, or contains the base sequence of SEQ ID NO: 11.

In one embodiment, the oligonucleic acid of (a-2′) has a sequence identity to SEQ ID NO: 12 (5′-cttaaatgacagagatacaatgt-3′) of at least approx. 60%, approx. 70%, approx. 80%, approx. 85%, approx. 90%, approx. 95%, approx. 96%, approx. 97%, approx. 98%, or approx. 99% or larger. In another embodiment, the oligonucleic acid (a-2′) is composed of a base sequence of SEQ ID NO: 12, in which one or several bases, typically two or three bases, are deleted, substituted, added or inserted. In one embodiment, the oligonucleic acid (a-2′) is composed of the sequence of SEQ ID NO: 12, or contains the base sequence of SEQ ID NO: 12.

In another embodiment, the oligonucleic acid that can disrupt the stem-loop structure in the 3′UTR of mouse Regnase-1 mRNA is at least either oligonucleic acid (b-1′) or (b-2′) below:
(b-1′) an oligonucleic acid 10 to 30 bases in length that hybridizes to a sequence having 10 to 30 consecutive bases including positions 381 to 383 in SEQ ID NO: 2, within a base sequence from positions 354 to 388 in SEQ ID NO: 2; and
(b-2′) an oligonucleic acid 10 to 30 bases in length that hybridizes to a sequence having 10 to 30 consecutive bases including positions 387 to 389 in SEQ ID NO: 2, within a base sequence from positions 382 to 416 in SEQ ID NO: 2.

In one embodiment, the oligonucleic acid (b-1′) has a sequence identity to SEQ ID NO: 13 (5′-atggtgcctaactagccggt-3′) of at least approx. 60%, approx. 70%, approx. 80%, approx. 85%, approx. 90%, approx. 95%, approx. 96%, approx. 97%, approx. 98%, or approx. 99% or larger. In another embodiment, the oligonucleic acid (b-1′) is composed of a base sequence of SEQ ID NO: 13, in which one or several bases, typically two or three bases, are deleted, substituted, added or inserted. In one embodiment, the oligonucleic acid (b-1′) is composed of the sequence of SEQ ID NO: 13, or contains the base sequence of SEQ ID NO: 13.

In one embodiment, the oligonucleic acid (b-2′) has a sequence identity to SEQ ID NO: 14 (5′-cctcagagagcaggcacatg-3′) of at least approx. 60%, approx. 70%, approx. 80%, approx. 85%, approx. 90%, approx. 95%, approx. 96%, approx. 97%, approx. 98%, or approx. 99% or larger. In another embodiment, the oligonucleic acid (b-2′) is composed of a base sequence of SEQ ID NO: 14, in which one or several bases, typically two or three bases, are deleted, substituted, added or inserted. In one mode and in one embodiment, the oligonucleic acid (b-2′) is composed of the sequence of SEQ ID NO: 14, or contains the base sequence of SEQ ID NO: 14.

In the prophylactic or therapeutic drug for PH of the present disclosure, the oligonucleic acid that can disrupt the stem-loop structure in the 3′UTR of Regnase-1 mRNA may be of a single type of sequence used independently, or may be of a combination of two or more types of sequence used in combination. In a case where two or more types of sequence are used as the oligonucleic acid, one composition may contain all of the oligonucleic acids, or each of two or more types of composition may individually contain one or more types of oligonucleic acids. The two or more kinds of oligonucleic acids, when contained in one composition, preferably do not form a complementary binding in between.

In one embodiment of the prophylactic or therapeutic drug for PH of the present disclosure intended for use in human, the oligonucleic acid that can disrupt the stem-loop structure in the 3′UTR of Regnase-1 mRNA may be used by combining one kind of the oligonucleic acid selected from (a-1) and (a-2), with one kind of the oligonucleic acid selected from (b-1) and (b-2). More specifically, exemplified are combination of the oligonucleic acids (a-1) and (b-1), combination of the oligonucleic acids (a-1) and (b-2), combination of the oligonucleic acids (a-2) and (b-1), and combination of the oligonucleic acids (a-2) and (b-2).

In one embodiment of the prophylactic or therapeutic drug for PH of the present disclosure intended for use in mouse, the oligonucleic acid that can disrupt the stem-loop structure in the 3′UTR of Regnase-1 mRNA may be used by combining one kind of the oligonucleic acid selected from (a-1′) and (a-2′), with one kind of the oligonucleic acid selected from (b-1′) and (b-2′). More specifically, exemplified are combination of the oligonucleic acids (a-1′) and (b-1′), combination of the oligonucleic acids (a-1′) and (b-2′), combination of the oligonucleic acids (a-2′) and (b-1′), and combination of the oligonucleic acids (a-2′) and (b-2′).

In the prophylactic or therapeutic drug for PH of the present disclosure, the oligonucleic acid that can disrupt the stem-loop structure in the 3′UTR of Regnase-1 mRNA may be composed of any of natural nucleotide, artificial nucleotide, or combination of one or more natural nucleotides and one or more artificial nucleotides. The natural nucleotide is exemplified by deoxyribonucleotide and ribonucleotide. The artificial nucleotide may only be selected from those having structures different from those of the natural nucleotide, and capable of enhancing nuclease resistance and binding affinity with target sequence. For example, usable are artificial nucleotides described in Deleavey, G. F., & Damha, M. J. (2012), Designing chemically modified oligonucleotides for targeted gene silencing, Chemistry & Biology, 19 (8), 937-954, the contents of this clarified source are incorporated herein by reference. Specific examples of the artificial nucleotide include abasic nucleoside; arabinonucleoside, 2′-deoxyuridine, α-deoxyribonucleoside, β-L-deoxyribonucleoside, and other nucleoside with modified sugar; peptide nucleic acid (PNA), peptide nucleic acid having phosphate group bound thereto (PHONA), crosslinked artificial nucleic acid (LNA), 2′-0,4′-C-ethylene-bridged nucleic acid (ENA), constrained ethyl (cEt), and morpholino nucleic acid. The other artificial nucleotide with modified sugar is exemplified by those having modified sugar that includes substituted pentose such as 2′-O-methylribose, 2′-O-methoxyethylribose, 2′-deoxy-2′-fluororibose, and 3′-O-methylribose; 1′,2′-deoxyribose; arabinose; substituted arabinose; hexose and α-anomer thereof. The artificial nucleotide with modified base is exemplified by those having pyrimidine such as 5-hydroxycytosine, 5-methylcytosine, 5-fluorouracil, and 4-thiouracil; purine such as 6-methyladenine and 6-thioguanosine; and other heterocyclic base. The artificial nucleotides in the oligonucleic acid may all be of the same type, or may be of two or more different types.

In the prophylactic or therapeutic drug for PH of the present disclosure, morpholino oligo is exemplified as a preferred example of the oligonucleic acid that can disrupt the stem-loop structure in the 3′UTR of Regnase-1 mRNA. The morpholino oligo is an oligonucleic acid having repeated therein a structure illustrated below:

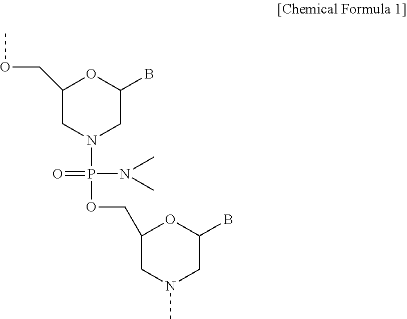

In the formula, B represents adenine, cytosine, guanine, or thymine, and each broken line indicates a point of binding with an adjacent structure.

In the prophylactic or therapeutic drug for PH of the present disclosure, single-stranded DNA is exemplified as a preferred example of the oligonucleic acid that can disrupt the stem-loop structure in the 3′UTR of Regnase-1 mRNA.

In a case where the oligonucleic acid is used as the substance that can disrupt the stem-loop structure in the 3′UTR of Regnase-1 mRNA in the prophylactic or therapeutic drug for PH of the present disclosure, such oligonucleic acid may be bound to one or more components, or to a conjugate that enhances the activity or uptake into cell. Such component includes, but not limited to, cholesterol components, cholic acid, thioether (e.g., hexyl-S-tritylthiol), thiocholesterol, aliphatic chain (e.g., dodecanediol or undecyl residues), phospholipids (e.g. di-hexadecyl-rac-glycerol or triethylammonium 1,2-di-O-hexadecyl-rac-glycero-3-H-phosphonate), polyamine, polyethylene glycol chain, adamantaneacetic acid, palmityl component, octadecylamine, hexylamino-carbonyl-t-oxycholesterol, and octaguanidine dendrimer component.

### [Target PH]

The prophylactic or therapeutic drug for PH of the present disclosure uses the substance that disrupts the stem-loop structure in the 3′UTR of Regnase-1 mRNA, in order to prevent or treat PH. In the present disclosure, the prophylactic drug for PH means a drug used for a subject at a risk of onset or recurrence of PH, in order to prevent onset or recurrence of PH, in order to reduce a risk of onset or recurrence of PH, or in order to delay onset or recurrence of PH. Meanwhile, in the present disclosure, the therapeutic drug for PH means a drug used for a subject with PH, in order to relieve or eliminate the cause for PH, in order to delay or stop progression of PH, and/or, in order to relieve, alleviate, improve, or eliminate the symptom of PH.

Types of PH to which the prophylactic or therapeutic drug for PH of the present disclosure is applied may be any of those of Group 1, Group 1′, Group 1″, Group 2, Group 3, Group 4, and Group 5 of the Nice Classification. Group 1 (PAH) is exemplified as a preferred type of PH to be aimed at. In one preferred embodiment of the prophylactic or therapeutic drug for PH of the present disclosure, I/HPAH and PCTD-PAH may be aimed at for their prevention or treatment.

### [Target Animal]

The target animal to which the prophylactic or therapeutic drug for PH of the present disclosure is administered may be, but not particularly limited to, mammal such as a human, mouse, rat, hamster, guinea pig, rabbit, cat, dog, goat, sheep, pig, cow or monkey. Human is preferred.

### [Dosage and Administration]

Method for administering the prophylactic or therapeutic drug for PH in the present disclosure is exemplified by, but not particularly limited to, oral administration, intravascular (intraarterial or intravenous) injection, pulmonary administration (inhalation), continuous infusion, subcutaneous administration, intramuscular administration, enteral administration, intraperitoneal administration, and topical administration. In one embodiment of the present disclosure, a preferred method for administering the prophylactic or therapeutic drug for PH is exemplified by pulmonary administration.

In connection with the dose of the prophylactic or therapeutic drug for PH, an effective amount for prevention and treatment may be appropriately set, typically depending on age, body weight, and type or clinical condition of PH of the target subject, or on the type of active ingredient to be used. For example, a daily dose of the substance that disrupts the stem-loop structure in the 3′UTR of Regnase-1 mRNA for adults may be appropriately set within a range from approx. 0.01 to 100 mg/kg, or from approx. 0.5 to 5 mg/kg. The prophylactic or therapeutic drug for PH may be administered once a day, or in several portions per day only for one day. Alternatively, the prophylactic or therapeutic drug for PH may be used continuously, at intervals of one day or several days.

### [Formulation]

The prophylactic or therapeutic drug for PH of the present disclosure is prepared and provided as a pharmaceutical composition with a desired dosage form, by blending the substance that disrupts the stem-loop structure in the 3′UTR of Regnase-1 mRNA, typically with transfection agent, stabilizer, excipient, antioxidant, buffer, preservative, surfactant, chelating agent, binder, sterile water, or physiological saline. The pharmaceutical composition may be formulated by any of known methods suited to the dosage form.

### Example

The present disclosure is by no means limited to the aforementioned Description of Embodiment, and Examples that follows. Various modifications that can be easily conceived by those skilled in the art without departing from the scope of the claims are also included in the present invention. Entire contents of the literatures cited herein will be incorporated by reference.

### 1. Experimental Materials and Methods

### (1) Human Sample

All experiments with human samples were conducted under the approval of the Institutional Review Board at the National Cerebral and Cardiovascular Center. Blood samples were obtained from 77 PH patients and 77 healthy volunteers (HV). Written consents on use of the blood samples for biological research have been obtained from all patients and healthy volunteers.

### (2) Isolation of Peripheral Blood Mononuclear Cell (PBMC)

From human peripheral blood collected in an EDTA blood collection tube, PBMC was isolated with use of Lymphoprep Tube (Cosmo Bio Co., Ltd), according to the instruction.

(3) Measurement of Regnase-1 mRNA Level in PBMC

Human PBMC was lysed with use of TRIzol (Invitrogen), and total RNA was extracted with use of PureLink RNA Mini Kit (Invitrogen) and PureLink DNase (Invitrogen). The total RNA was subjected to qRT-PCR with use of PrimeScript RT reagent kit (Takara Bio) and TB Green Premix Ex Taq (trademark) II (Takara Bio). After reverse transcription according to the manufacturer's manual, the product was subjected to qPCR under conditions of 95° C. for 30 seconds, then under 95° C. for 5 seconds and 60° C. for 30 seconds, repeated 45 cycles. Fluorescence data was collected and analyzed with use of LightCycler 96 (Roche). Genes and primers thereof used herein are as follows.

Regnase-1_f:
(SEQ ID NO: 15)
5′-GAAGAGGAAAAGGAGGGCAG-3′
Regnase-1_r:
(SEQ ID NO: 16)
5′-CTCCAGGATGGCACAAACAC-3′
hGAPDH_f:
(SEQ ID NO: 17)
5′-ATGGGGAAGGTGAAGGTCG-3′
hGAPDH_r:
(SEQ ID NO: 18)
5′-GGGGTCATTGATGGCAACAATA-3′

### (4) Experimental Animals

All experiments were conducted with use of 8- to 14-week-old mice. Zc3h12a^{flox/flox} mouse having flox sites introduced into ZC3H12A gene (encoding Regnase-1);
CD11c-Cre mouse having introduced therein a DNA recombinant enzyme Cre induced by CD11c gene promoter;
LysM-Cre mouse having introduced therein a DNA recombinant enzyme Cre induced by lysozyme gene promoter;
Rag2^{-/-} mouse having Rag2 knocked out; and
Reg1f/+ mouse having a Regnase-1 heterozygous in bone marrow cell, were prepared.

The Zc3h12a^{flox/flox} mouse and the CD11c-Cre mouse was further crossed to create a CD11c-Cre⁺Zc3h12a^{fl/fl} mouse deficient in Regnase-1 in alveolar macrophage and in dendritic cell (cDC). The Zc3h12a^{flox/flox} mouse was also crossed with the LysM-Cre mouse to create Lysm^{Cre/+}Zc3h12a^{fl/fl} mouse deficient in Regnase-1 in alveolar macrophage and in neutrophil.

The CD11c-Cre⁺Zc3h12a^{fl/fl} mouse was further crossed with a Rag2^{-/-} mouse to create a CD11c-Cre⁺Zc3h12a^{fl/fl}Rag2^{-/-} mouse deficient in T cell and B cell. Also the Lysm^{Cre/+}Zc3h12a^{fl/fl} mouse was further crossed with the Rag2^{-/-} mouse to create a Lysm^{Cre/+}Zc3h12a^{fl/fl}Rag2^{-/-} mouse deficient in T cell and B cell.

All undescribed experiments were conducted with use of C57BL/6 mice purchased from CLEA Japan. All mice were fed on general diets for mouse and water, in a pathogen-free environment at 24±1° C., under a 12-h/12-h light/dark cycle. All animal experiments were conducted under the approval of Kyoto University Animal Experiment Board (approval number: MedKyo21057) and the National Cerebral and Cardiovascular Center.

### (5) Creation and Analysis of PH Model Mice

Wild-type C57BL/6 mice at 8 weeks of age were placed in a hypoxic chamber with 10% oxygen, and bred therein for 4 days to induce clinical condition of PH. After the 4-day breeding, the mice were subjected to bronchoalveolar lavage to collect a bronchoalveolar lavage fluid. The obtained bronchoalveolar lavage fluid was processed to obtain a cell fraction, and the expression levels of IL-6 and Regnase-1 were measured by RT-qPCR.

Besides that, monocrotaline at 60 mg/kg was subcutaneously administered to a 6-week-old female SD rat, to induce clinical condition of PH. After 4 weeks from the administration of monocrotaline, the right ventricular systolic pressure (RVSP) and the Fulton coefficient (weight ratio of right ventricle/(left ventricle+septum)) were measured. RNA was further extracted from the lung tissue, and the expression levels of IL-6 and Regnase-1 were measured by RT-qPCR.

### (6) Analysis of Mice Deficient in Regnase-1 in Alveolar Macrophage

Nine-week-old CD11c-Cre⁺Zc3h12a^{fl/fl} mice and Zc3h12a^{flox/flox} mice, or 13-week-old Lysm^{Cre/+}Zc3h12a^{fl/fl} mice and Zc3h12a^{flox/flox} mice were subjected to hemodynamic measurement for pathological analysis of pulmonary hypertension. After the hemodynamic measurement, each mouse was sacrificed, from which the heart and the lungs were excised and weighed, followed by tissue analysis.

(7) Chronic Hypoxic Load Experiment on Reg1 f/+ Mice and CD11c-Cre⁺Zc3h12a^{fl/+} Mice

Eight-week-old Reg1 f/+ mice or CD11c-Cre⁺Zc3h12a^{fl/+} mice were bred under hypoxic load for 4 weeks. The breeding under hypoxic load was conducted by keeping the animals in a hypoxic chamber with 10% oxygen throughout 4 weeks, except that the chamber was opened once a week for approx. 5 minutes for cleaning. The hypoxic gas was continuously fed into the hypoxic chamber at a flow rate of 7 L/min. The mice after kept under hypoxic load were subjected to hemodynamic measurement, then sacrificed, from which the heart and the lungs were excised and weighed, followed by tissue analysis.

(8) Therapeutic Experiment of PAH Model Mouse with Use of Anti-IL-6 Receptor Neutralizing Antibody

The anti-IL-6 receptor neutralizing antibody used herein was MR16-1, which is a rat monoclonal IgG antibody against mouse IL-6 receptor provided by Chugai Pharmaceutical Co., Ltd. Two milligrams of MR16-1 or a control antibody (rat non-immune isotype IgG; MP Biomedicals, Solon, OH) was administered to CD11c-Cre⁺Zc3h12a^{fl/fl} mice by intravenous injection from the age of 4 weeks. Thereafter, 0.5 mg of MR16-1 or the control antibody was intraperitoneally administered once a week. The animals at the age of 8 weeks were subjected to hemodynamic measurement, then sacrificed, from which the heart and the lungs were excised and weighed, followed by tissue analysis.

(9) Therapeutic Experiment of PAH Model Mouse with Use of PDGF Receptor Inhibitor

Imatinib was used as a PDGF receptor inhibitor. Imatinib was administered to CD11c-Cre⁺Zc3h12a^{fl/fl} mice in a mixed feed, with the dose adjusted to 50 mg/kg/day at the age of 4 weeks and thereafter. The control group was allowed to freely take a powdered food. After subjected to the hemodynamic measurement at the age of 8 weeks, the animals were sacrificed, from which the heart and the lungs were excised and weighed, followed by tissue analysis.

(10) Therapeutic Experiment of PAH Model Mouse with Use of IL-1 Receptor Inhibitor

Anakinra was used as an IL-1 receptor inhibitor. Anakinra was intraperitoneally administered to CD11c-Cre⁺Zc3h12a^{fl/fl} mice, while adjusting the dose to 20 mg/kg/day at the age of 4 weeks and thereafter. The control group was allowed to freely take a powdered food. After subjected to the hemodynamic measurement at the age of 8 weeks, the animals were sacrificed, from which the heart and the lungs were excised and weighed, followed by tissue analysis.

(11) Therapeutic Experiment of PAH Model Mouse with Use of Morpholino Oligo (MO) that Suppresses Decomposition of Regnase-1

As a mouse Regnase-1 morpholino-modified oligonucleic acid (Reg1 MO) that suppresses decomposition of mouse Regnase-1, used herein was a Regnase-1/morpholino oligo mixture that contains equimolar amounts of 191-210MO and 378-392MO below. 191-210MO Has a base sequence complementary to positions 201 to 223 of the base sequence of SEQ ID NO: 2 (3′UTR of mouse Regnase-1 mRNA). 378-392MO Has a base sequence complementary to positions 384 to 403 of the base sequence of SEQ ID NO: 2. A control used herein was a morpholino oligo (control oligo) composed of the base sequence below.

191-210MO:
(SEQ ID NO: 12)
5′-cttaaatgacagagatacaatgt-3′
378-392MO:
(SEQ ID NO: 14)
5′-cctcagagagcaggcacatg-3′
Control oligo:
(SEQ ID NO: 19)
5′-cctcttacctcagttacaatttata-3′

Reg1 MO or a control oligo was intratracheally administered to male C57BL/6 mice at an age of 8 weeks, once a week and three times in total, with the dose adjusted to 25 μg/week (50 μL administration). One day after the first administration, the mice were placed in a hypoxic chamber with 10% oxygen, and bred therein for 4 weeks. Four weeks after, the mice after kept under hypoxic load were subjected to hemodynamic measurement, then sacrificed, from which the heart and the lungs were excised and weighed, followed by tissue analysis.

(12) Hemodynamic Measurement with Right Heart Catheter

Mice were anesthetized with isoflurane (1.5 to 2%). During the operative procedure, the body temperature of the mice was maintained at 37 to 38° C. with a thermostatically controlled heat pad coupled with a rectal temperature monitor. The mice were subjected to tracheostomy, and ventilated with a respirator (VentElite; Harvard Apparatus) at a tidal volume of 200 to 300 μL, and a rate of 160 to 180 breath/min.

A polyethylene tube (SP-31) was inserted into the right external jugular vein and advanced to the right ventricle, to measure the right ventricular pressure (RVP). A polyethylene tube (PE50) with the tip stretched and thinned under heating was inserted into the right carotid artery, to measure the arterial pressure (AP). AP and RVP signals were detected with a pressure transducer (MLT0670; AD Instruments), relayed with a pressure amplifier (ML117; AD Instruments), continuously sampled with Power Lab system (AD Instruments, Colorado Springs, CO), and recorded by a computer with use of Chart software (AD Instruments). The heart rate was calculated from systolic peaks of the arterial pressure. In the experiment, the measurement was conducted only under conditions where the mean arterial pressure is 50 mmHg or above, and the heart rate falls in the range from 350 to 600 times/min. The measurement was not conducted under conditions where the mean arterial pressure drops below 50 mmHg, or the heart rate drops to 350 times/min or below.

### (13) Morphological Analysis

After the hemodynamic measurement with the right heart catheter, the mice were euthanized by exsanguination from the inferior vena cava. After the euthanasia, PBS was perfused through the right heart catheter, and the left atrium was incised to remove the blood. The heart was removed, the atria was removed, and the right ventricle (RV) was separated from the left ventricle (LV) and the septum. The tissues were weighed to determine weight ratio of the right ventricle and the left ventricle (RV/(LV+septum)) called Fulton coefficient, which was used as an index for right ventricular hypertrophy.

The lungs were collected for histological analysis. The trachea was perfused with 4% paraformaldehyde (PFA), and fixed under a condition that allows the airway to stretch. The resected lung samples were fixed overnight at 4° C. in 4% PFA, replaced with PBS, embedded in paraffin, and sectioned at 4 μm thickness. Morphology of the pulmonary blood vessel was analyzed under Elastica van Gieson staining and hematoxylin-eosin staining. Images of the pulmonary blood vessels were photographed with ScanScope CS (Leica) or NanoZoomer (Hamamatsu Photonics). The lung sections from the animals, randomly selected from the individual experimental groups, were subjected to morphological analysis. The pulmonary vascular remodeling was examined by measuring the medial wall thickness coefficient (% wall thickness) for the pulmonary artery in the lung parenchyma, the small artery in the terminal bronchiole level, and the arteriole in the lung acinus level. The medial wall thickness coefficient was estimated by doubling the distance between the internal elastic lamina and the external elastic lamina, dividing the product by the distance between the external elastic laminae (diameter of blood vessel), and multiplying the quotient by 100. For the blood vessel having only one layer of elastic lamina, the distance between the elastic lamina and the basement membrane under the endothelium was measured. The medial wall thickness was analyzed only for blood vessels sectioned into a near-circular form. The diameter of the pulmonary artery was measured with use of Aperio ImageScope (Leica). The medial wall thickness coefficient was estimated from at least 15 small pulmonary arteries and small pulmonary arterioles per mouse.

### (14) Immunohistochemical Analysis

The tissues were immunostained with use of anti-von Willebrand Factor (vWF) polyclonal antibody (bs-0586R; Bioss Inc.), and anti-α-smooth muscle actin (α-SMA) antibody (sc-32251; Santa Cruz). In order to eliminate any reaction involving the endogenous murine immunoglobulin in the tissues, Histofine-MOUSESTAIN KIT (Nichirei) was used as per the manufacturer's manual.

### (15) Preparation of Lung-Derived Single Cell Suspension

The mouse lung sample was minced with scissors, and digested in a RPMI medium containing 100 μg/ml DNase I (Roche Applied Science) and 50 μg/ml Liberase TM (Roche Applied Science) at 37° C. for 40 min. Next, 500 mM EDTA (Nacalai Tesque) was added so as to adjust the final concentration to 10 mM, and the mixture was incubated at 37° C. for 10 minutes. After the digestion, the sample was processed with GentleMACS (trademark) Dissociator (Miltenyi Biotec), and filtered through a 40 μm cell strainer. The filtered sample was then centrifuged, the supernatant was removed, and the cell pellet was lyzed with an ACK solution to remove the red blood cell. The thus processed sample was washed with RPMI medium containing 10% fetal bovine serum, the mixture was centrifuged to collect the cell pellet, which was then subjected to flow cytometry.

### (16) Flow Cytometry

The cells were stained with Fixable Viability Dye eFluor 506 (eBiosciences) for live/dead exclusion, and then treated with anti-mouse CD16/CD32 antibody, as per the manufacturer's manual. After blocking Fc receptor, the cells were incubated on ice, with any of surface antigen-staining antibodies for 20 minutes. The antibodies used herein are FITC-conjugated anti-mouse CD4 antibody (GK1.5, Biolegend);
FITC-conjugated anti-mouse CD19 antibody (clone 6D5, Biolegend);
PE-conjugated anti-mouse SiglecF antibody (E50-2440, BD Pharmingen);
PE-conjugated anti-mouse B220 antibody (RA3-6B2, Biolegend);
APC-conjugated anti-mouse CD11c antibody (N418, Biolegend);
Alexa Fluor 700-conjugated anti-mouse Ly-6G antibody (1A8, Biolegend);
Alexa Fluor 700-conjugated anti-mouse I-A/I-E antibody (M5/114.15.2, Biolegend);
APC-Cy7-conjugated anti-mouse CD11b antibody (M1/70, Biolegend);
APC-Cy7-conjugated anti-mouse SiglecF antibody (E50-2440, BD Pharmingen);
PE-Cy7-conjugated anti-mouse Ly-6 C antibody (HK1.4, Biolegend);
PE-Cy7-conjugated anti-mouse CD11b antibody (M1/70, Biolegend);
PerCP-Cy5.5-conjugated anti-mouse CD45.2 antibody (clone 104, Biolegend); and
BV421-conjugated anti-mouse CD64 antibody (X54-5/7.1, Biolegend). Data were acquired with use of LSRFotessa X-20 (BD Biosciences), and sorted with use of FACSAria III (BD Biosciences). Data were analyzed with use of FlowJo 10.5.3 software (FlowJo, LLC).

### (17) RT-qPCR

The cells or the tissues were lysed with TRIzol reagent (Invitrogen), to extract the total RNA. The total RNA was reverse-transcribed with use of ReverTra Ace qPCR RT Master Mix and gDNA remover (Toyobo), as per the manufacturer's manual. The cDNA fragment was amplified with use of SYBR Green PCR Master Mix (Applied Biosystems), and fluorescence was detected with use of StepOnePlus Real-Time PCR System (Applied Biosystems). Mouse Actb mRNA level was used for normalization.

### (18) Laser Microdissection

Frozen sections of the lung tissues were placed on glass slides, stained with toluidine blue, and then immersed in 100% ethanol. The intrapulmonary arteries with a diameter of 250 to 500 μm were selected, and then isolated with use of Laser Microbeam System (Leica Microsystems, Germany).

(19) mRNA Extraction

mRNA was isolated according to the Chomczynski protocol with some modifications. After washing, RNA was resuspended in 10 μL purified water (RNase free), and digested with DNase (Ambion, Austin, TX; 1 U, 30 min, 37° C.). Extraction was then repeated, and finally the RNA was resuspended in 4 μL purified water.

### (20) RNA Sequence and Bioinformatics Analysis

Macrophages isolated from the pulmonary arteries and alveoli of the CD11c^{Cre/+}Zc3h12a^{fl/fl} mice were lysed with TRizol reagent, to extract the total RNA as described previously. After purification with use of RNA Clean & Concentrator-5 (Zymo Research), the quality of the RNA was checked with use of 2100 Bioanalyzer RNA 6000 Nano assay or RNA 6000 Pico assay (Agilent). Each sample before sequencing was found to have an RIN value (RNA integrity number) of larger than 7.0. cDNA libraries were created with use of NEBNext Ultra II Directional RNA Library Prep Kit (Illumina) according to the manufacturer's manual, and sequenced with use of NextSeq500 (Illumina, 75 cycles). Gene ontology (GO) enrichment analysis was conducted with use of PANTHER (Protein ANalysis THrough Evolutionary Relationships) database, with reference to GO terms related to biological processes of Mus musculus species.

### (21) Luciferase Reporter Assay

Regnase-1 (WT), Regnase-1 mutant (D141N), or empty (control) expression plasmid was transfected to HEK293 cell, together with luciferase reporter plasmid pGL3 that contains the 3′UTR of a predetermined gene. After 24-hour culture, the cells were lysed, and luciferase activity in the lysate was measured with use of Dual-Luciferase Reporter Assay System (Promega). As an internal standard, also a gene that encodes renilla luciferase was concurrently transfected.

### (22) Statistical Analysis

All data are expressed in the form of mean±standard deviation. The significance test among the multiple groups was conducted by one-way ANOVA, followed by the Turkey-Kramer test. The test between two groups was conducted by the Student's t-test. Statistical significance was judged by a P-value of smaller than 0.05. * Stands for P<0.05, ** for P<0.01, *** for P<0.001, and **** for P<0.0001.

### 2. Experimental Results

### 2-1. PH Marker

### (1) Regnase-1 Expression Level in PBMC of PH Patients

In order to examine involvement of Regnase-1 in PH, the Regnase-1 mRNA level in PBMC was compared between PH patients and healthy volunteers (HVs), with the age and the sex matched in between. Details of the age, sex, disease type, Regnase-1 expression level of and the like of the PH patients and the healthy volunteers are summarized in Table 1.

TABLE 1

| | HV | PH Patients | ZC3H12A mRNA expression level | | | |
|---|---|---|---|---|---|---|
| | (n-77) | (n-77) | 1st quartile | 2nd quartile | 3rd quartile | 4th quartile |
| ZC3H12A mRNA expression level | 0.01003 | 0.00680 | 0.00385 | 0.00615 | 0.00776 | 0.01060 |
| | (0.00855- | (0.00493- | (0.00319- | (0.00532- | (0.00744- | (0.00969- |
| | 0.01222) | 0.00907) | 0.00410) | 0.00648) | 0.00809) | 0.01184) |
| Age, years | 44 | 48 | 54 | 42 | 49 | 48 |
| | (32-52) | (36-59) | (28-63) | (34-54) | (40-63) | (37-58) |
| Sex, female n (%) | 52 | 58 | 11 | 16 | 16 | 15 |
| | (68) | (75) | (58) | (80) | (84) | (79) |

PH subgroups

| Idiopathic or heritable PAH | | — | 33 | 8 | 12 | 4 | 9 |
|---|---|---|---|---|---|---|---|
| (I/HPAH), n | | | | | | | |
| Connective tissue disease-derived | | — | 22 | 4 | 4 | 7 | 7 |
| PAH (CTD-PAH), n | | | | | | | |
| Congenital heart disease-derived | | — | 6 | 0 | 2 | 4 | 0 |
| PAH (CHD-PAH), n | | | | | | | |
| Portal vein pulmonary hypertension | | | 5 | 0 | 1 | 2 | 2 |
| (PoPH), n | | | | | | | |
| Drug-induced PAH, n | | — | 2 | 3 | 1 | 0 | 0 |
| Chronic thromboembolic pulmonary | | — | 4 | 0 | 0 | 1 | 1 |
| hypertension (CTE-PH), n | | | | | | | |
| Nice classification group 3 and | | — | 5 | 4 | 0 | 1 | 0 |
| group 5, n | | | | | | | |
| Nice classification group 3, n | | — | 3 | 2 | 0 | 1 | 0 |
| Down's syndrome, n | | — | 1 | 1 | 0 | 0 | 0 |
| Castleman's disease, n | | — | 1 | 1 | 0 | 0 | 0 |
| WHO functional | Class 1 | | 2 | 1 | 0 | 0 | 1 |
| classification, | | | (2.5) | (5) | (0) | (0) | (5) |
| n (%) | Class 2 | — | 30 | 3 | 11 | 6 | 10 |
| | | | (39) | (16) | (55) | (32) | (53) |
| | Class 3 | — | 43 | 13 | 9 | 13 | 8 |
| | | | (56) | (68) | (45) | (68) | (42) |
| | Class 4 | — | 2 | 2 | 0 | 0 | 0 |
| | | | (2.5) | (11) | (0) | (0) | (0) |

| | | | | | | |
|---|---|---|---|---|---|---|
| Serum uric acid level, mg/dL | — | 5.8 ± 1.5 | 6.9 ± 1.8 | 5.6 ± 1.8 | 5.6 ± 0.7 | 5.0 ± 1.0 |
| Serum CRP level, mg/dL | — | 0.09 | 0.15 | 0.10 | 0.12 | 0.05 |
| | | (0.03-0.35) | (0.02-0.69) | (0.02-0.35) | (0.04-0.45) | (0.02-0.14) |
| Serum BNP level, pg/mL | — | 43.2 | 103.8 | 47.9 | 73.7 | 34.6 |
| | | (16.9-124.6) | (13.0-227.1) | (19.6-118.1) | (14.0-167.0) | (18.0-54.1) |
| 6-Minute walk distance^{#}, m | — | 471 ± 116 | 467 ± 147 | 469 ± 103 | 447 ± 112 | 498 ± 119 |
| Right atrial pressure, mmHg | — | 4.0 | 4.0 | 3.5 | 3.0 | 3.0 |
| | | (2.0-5.0) | (3.0-5.0) | (2.0-6.0) | (0.0-6.0) | (2.0-5.0) |
| Mean pulmonary arterial pressure, | — | 39 ± 14 | 44 ± 14 | 37 ± 11 | 41 ± 14 | 35 ± 14 |
| mmHg | | | | | | |
| Pulmonary vascular resistance, | — | 7.0 | 7.0 | 6.7 | 8.6 | 5.9 |
| Wood units | | (5.3-10.7) | (5.5-12.1) | (4.4-10.2) | (5.7-14.5) | (3.9-9.0) |
| Cardiac index, l/min/m² | — | 2.6 ± 0.8 | 2.6 ± 0.9 | 2.7 ± 0.9 | 2.3 ± 0.4 | 2.7 ± 0.6 |
| Right ventricular ejection fraction⁺, | — | 38.9 ± 10.8 | 33.1 ± 11.6 | 38.8 ± 10.9 | 40.5 ± 10.3 | 43.3 ± 8.8 |
| % | | | | | | |
| Number of patients under PAH- | — | 53 | 11 | 14 | 12 | 16 |
| specific drug therapy | | (68) | (58) | (70) | (63) | (84) |
| Number of complex events | — | 12 | 8 | 1 | 2 | 1 |
| | | (16) | (42) | (5) | (11) | (5) |

Continuous variables are given by mean ± SD, or median (interquartile range), and categorical variables are given by the number and ratio (%) of patients. P-values of ZC3H12A expression level, age and female between HV and PH patients are P < 0.0001 (unpaired Student t-test), P = 0.10 (Welch test), and P = 0.28 (χ² test), respectively.
^{#}Results from n = 61 (1st quartile: 11, 2nd quartile: 19, 3rd quartile: 15, and 4th quartile 16).
⁺Results from n = 56 (1st quartile: 15, 2nd quartile: 12, 3rd quartile: 13, and 4th quartile 16).

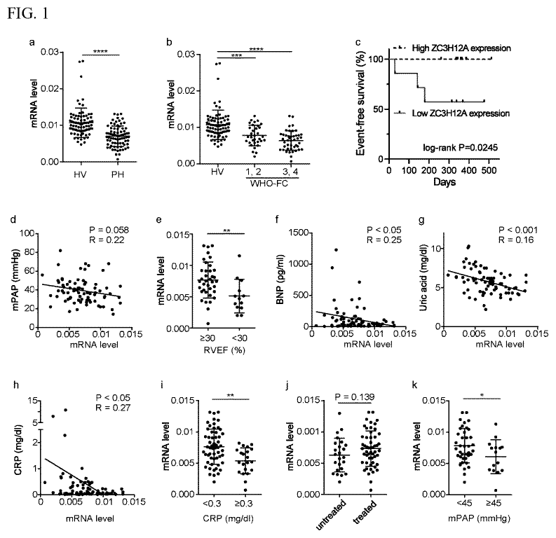

In the PH patients, the Regnase-1 expression level was found to significantly decrease (Table 1, FIG. 1(a)). The Regnase-1 expression level was found to be low, not only in the PH patients classified into severe classes (classes 3 and 4) by the WHO functional classification (WHO-FC), but also in the PH patients classified into mild classes (classes 1 and 2) (Table 1, FIG. 1(b)). While dividing the PH patients into two groups according to the Regnase-1 expression level, the patients with low Regnase-1 expression levels (n=7) demonstrated higher incidence of prognostic events such as death, heart failure, and lung transplantation, as compared with the patients with high Regnase-1 expression levels (n=10), proving notably poor prognosis (FIG. 1(c)). This indicates that the Regnase-1 expression level is positively correlated with improvement in the prognosis of the PH patients. The Regnase-1 expression level in PBMC was found to decrease not only in PAH patients, but also in Group 4 (CTEPH) and Group 3 (others) of the WHO functional classification, Down's syndrome, and Castleman's disease.

The Regnase-1 expression level was also found to inversely correlated with the mean pulmonary arterial pressure (mPAP) (FIG. 1(d)). The Regnase-1 expression level in the group with the right ventricular ejection fraction of smaller than 30% was found to significantly decrease, as compared with the group with that of 30% or large (FIG. 1(e)). Also the serum levels of brain natriuretic peptide (BNP) as a severity biomarker for heart failure, and uric acid (UA) as a severity marker of PH, were found to inversely correlated with the Regnase-1 mRNA level in PBMC of the PAH patients (FIGS. 1(f) and 1(g)), proving that the Regnase-1 expression level correlates with the severity of PAH. The Regnase-1 expression level was also found to reversely correlate with serum C-reactive protein (CRP) level, which is an inflammation marker (FIGS. 1(h) and 1(i)).

Furthermore, the PH patients were divided into two groups of patients (n=53) having been treated for PH and patients (n=24) not having been treated for pH, and then subjected to analysis of the Regnase-1 mRNA level in PBMC. The group treated for PH demonstrated a higher tendency of the Regnase-1 expression level, as compared with the group not treated for PH (FIG. 1(j)).

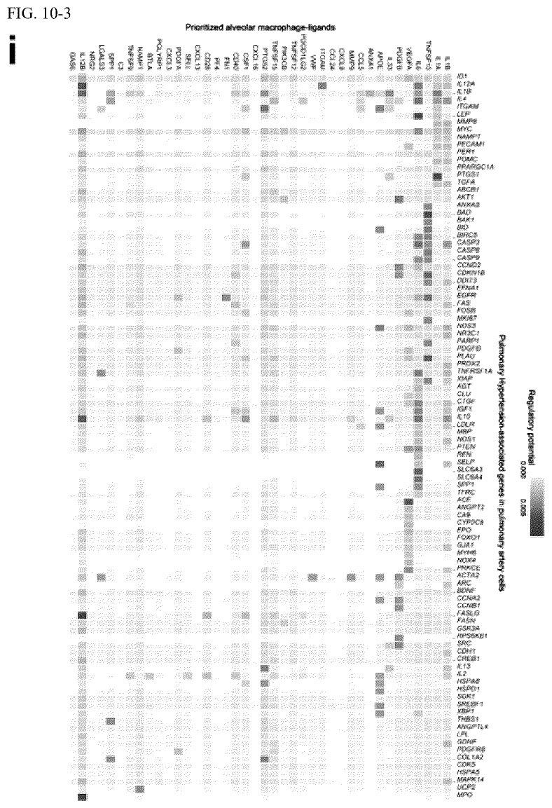

In addition, the PH patients (n=53) having been treated for PH were divided into a high mPAP group (45 mmHg or above: n=15) and a low mPAP group (below 45 mmHg: n=38), and then subjected to analysis of the Regnase-1 mRNA level in PBMC. The low mPAP group was found to demonstrate the Regnase-1 expression level significantly higher than that in the high mPAP group (FIG. 10)), proving that the PH patient group with treatment tolerance demonstrates the Regnase-1 expression level significantly lower that of the pH patient group with treatment responsiveness.

### (2) Regnase-1 Expression Level in PBMC, by PH Subgroups

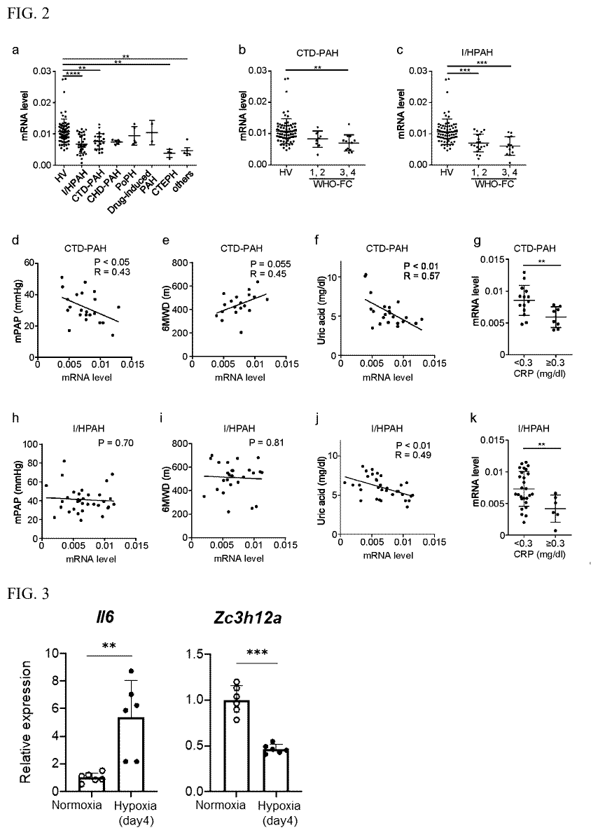

The Regnase-1 expression level was examined for each subgroup of PH. The Regnase-1 expression level was found to significantly decrease in various types of PH including connective tissue disease (CTD)-PAH, idiopathic or hereditary PAH (I/HPAH), and congenital heart disease (CHD)-PAH (FIG. 2(a)). The Regnase-1 expression level was found to be lower in the CTD-PAH patients and I/HPAH patients regardless of the WHO functional classification as compared with HV (FIGS. 2(b) and 2(c)). While the CTD-PAH patients demonstrated inverse correlation between the Regnase-1 expression level and mPAP (FIG. 2(d)), the I/HPAH patients did not demonstrate correlation between the Regnase-1 expression level and mPAP (FIG. 2(h)). Similarly, the Regnase-1 expression level was found to correlate with the six-minute walk distance in the CTD-PAH patients (FIG. 2(e)), meanwhile this correlation was not observed in the I/HPAH patients (FIG. 2(i)). On the other hand, the serum uric acid level was found to correlate with the Regnase-1 expression level both in CTD-PAH and I/HPAH (FIGS. 2(f) and 2(j)). When focused on the patients with high serum CRP level, the Regnase-1 expression level was found to be low both in the CTD-PAH patients and the I/HPAH patients (FIGS. 2(g) and 2(k)).

The results clarified that the Regnase-1 expression level in PBMC is usable as a diagnostic index for presence/absence of PH in various types of PH patients, particularly as a diagnostic index for the severity of PAH (particularly CTD-PAH).

The prognosis of PH (death, lung transplantation, hospitalization) was examined by univariate analysis and multivariate analysis. Results are summarized in Table 2. The results clarified that the Regnase-1 expression was found to serve as a factor related to prognosis, independently of other factors such as age, BNP, six-minute walk distance, right atrial pressure, mean pulmonary arterial pressure, and pulmonary vascular resistance.

TABLE 2

| Univariate analysis | HR | 95% CI | P-value | n | Event |
|---|---|---|---|---|---|
| ZC3H12A mRNA | 0.47 | 0.58-0.92 | 0.11 | 77 | 12 |
| expression level × 10³ | | | | | |
| Age, years | 1.04 | 1.00-1.09 | 0.045 | 77 | 12 |
| Serum BNP level, pg/ml | 1.002 | 1.000-1.004 | 0.006 | 77 | 12 |
| 6-Minute walk distance^{#}, | 0.992 | 0.984-0.999 | 0.038 | 61 | 5 |
| m | | | | | |
| Right atrial pressure, | 1.16 | 0.94-1.39 | 0.133 | 77 | 12 |
| mmHg | | | | | |
| Mean pulmonary arterial | 1.05 | 1.01-1.09 | 0.019 | 77 | 12 |
| pressure, mmHg | | | | | |
| Pulmonary vascular | 1.05 | 0.96-1.13 | 0.236 | 77 | 12 |
| resistance, Wood units | | | | | |
| Cardiac index, 1/min/m² | 1.12 | 0.50-2.17 | 0.770 | 77 | 12 |

| Multivariate analysis | | | | | | |
|---|---|---|---|---|---|---|
| ZC3H12A mRNA | | | | | | |
| expression level × 10³ | HR | 95% CI | P-value | n | Event | Covariate |
| Model 1 | 0.71 | 0.55-0.90 | 0.024 | 77 | 12 | Age |
| Model 2 | 0.76 | 0.59-0.97 | 0.032 | 77 | 12 | Serum BNP level |
| Model 3 | 0.66 | 0.43-0.98 | 0.049 | 61 | 5 | 6-Minute walk distance |
| Model 4 | 0.72 | 0.55-0.92 | 0.012 | 77 | 12 | Right atrial pressure |
| Model 5 | 0.74 | 0.59-0.93 | 0.012 | 77 | 12 | Mean pulmonary |
| | | | | | | arterial pressure |
| Model 6 | 0.73 | 0.57-0.92 | 0.011 | 77 | 12 | Pulmonary vascular |
| | | | | | | resistance |
| Model 7 | 0.74 | 0.58-0.93 | 0.011 | 77 | 12 | Cardiac index |

### 2-2. PAH Model Mice

### (1) Regnase-1 Expression Level in PH Model Mice

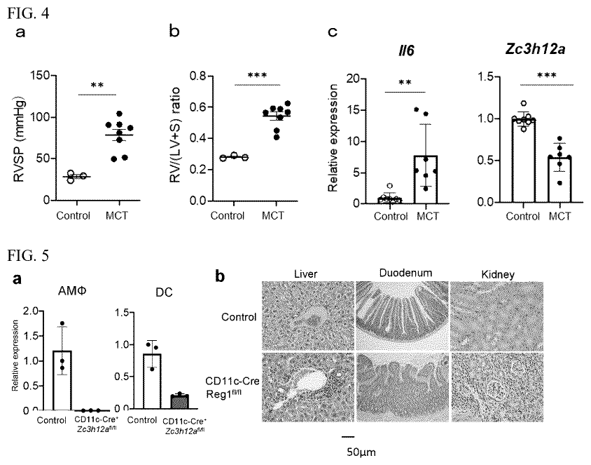

Wild-type mice (C57BL/6) were placed in a hypoxic chamber with 10% oxygen, and bred therein for 4 days to induce clinical condition of hypoxic PH. After the 4-day breeding, the mice were subjected to bronchoalveolar lavage to collect a bronchoalveolar lavage fluid. The obtained bronchoalveolar lavage fluid was processed to obtain a cell fraction, and the expression levels of IL-6 and Regnase-1 were measured by RT-qPCR. The results clarified that the mice, having the clinical condition of PH induced therein under hypoxia load, were found to demonstrate significant increase in the IL-6 expression level in the cells recovered from the bronchoalveoli, meanwhile significant decrease in the Regnase-1 expression level, as compared with the control mouse bred under normoxia (FIG. 3).

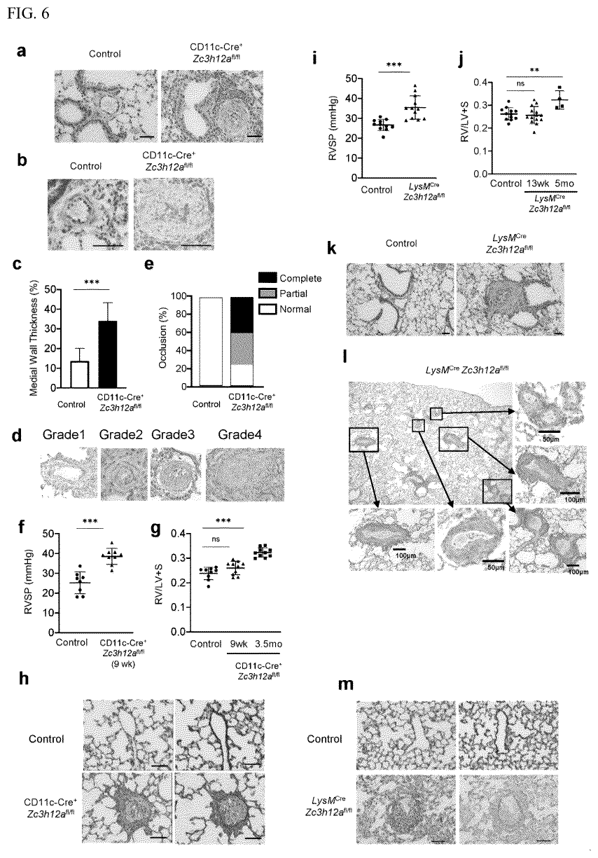

Also monocrotaline was administered to SD rats, to induce clinical condition of PH. After 4 weeks from the administration of monocrotaline, the right ventricular systolic pressure (RVSP) and the Fulton coefficient (weight ratio of right ventricle/(left ventricle+septum)) were measured. RNA was further extracted from the lung tissue, and the expression levels of IL-6 and Regnase-1 were measured by RT-qPCR. The mice after 4 weeks from the administration of monocrotaline were found to elevate the right ventricular systolic pressure (RVSP), and to elevate the Fulton coefficient (weight ratio of right ventricle/(left ventricle+septum)), which is an index of the degree of right ventricular hypertrophy, as compared with the control mice without administration of monocrotaline, thus confirmed that the clinical condition of PH was demonstrated (FIGS. 4(a) and 4(b)). The mice with administration of monocrotaline were found to significantly increase the IL-6 expression level in the lung tissues, and to significantly decrease the Regnase-1 expression level, as compared with the control mice (FIG. 4(c)).

Since two different species of the PH animal model demonstrated significant decrease in the Regnase-1 expression level, the results support that Regnase-1 is usable as a diagnostic index for PAH.

### (2) Analysis of Mice Deficient in Regnase-1 in Alveolar Macrophage

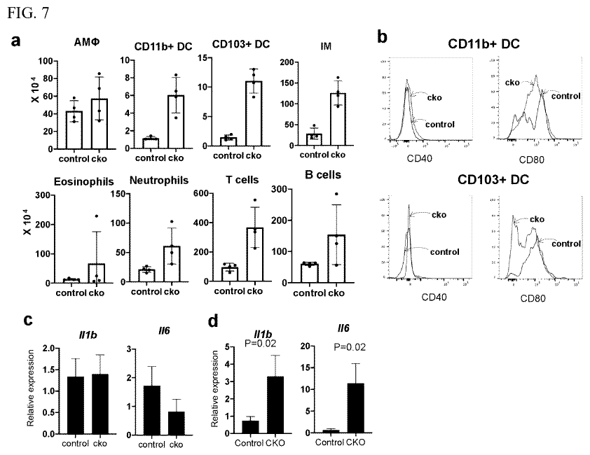

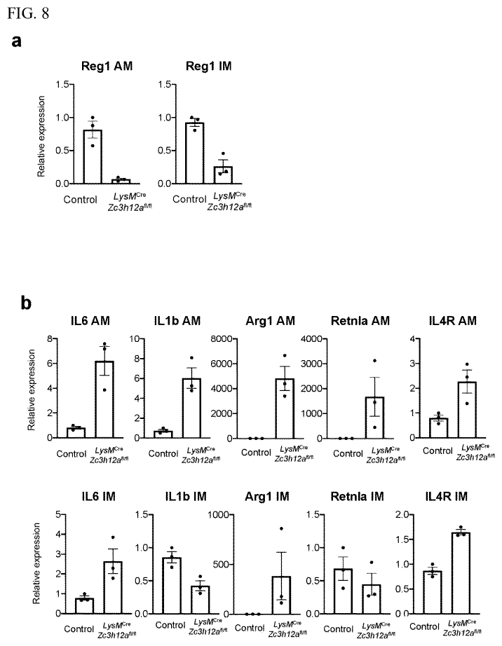

Next, whether or not Regnase-1 is involved in the pathogenesis of PAH was examined. Expression of Regnase-1 in bone marrow cell that highly produces inflammatory cytokine would modulate PAH. Therefore, first, CD11c-Cre⁺Zc3h12a^{fl/fl} mice deficient in Regnase-1 in dendritic cell (cDC) and alveolar macrophage were created (FIG. 5(a)). While immune cells were found to infiltrate into various organs in CD11c-Cre⁺Zc3h12a^{fl/fl} mice (FIG. 5(b)), it was confirmed from histological and immunohistochemical analyses that the pulmonary vascular remodeling in CD11c-Cre⁺Zc3h12a^{fl/fl} mice was characterized by medial wall thickening and interstitial infiltration of inflammatory cells (FIGS. 6(a) to 6(c)). Furthermore, histological changes that correspond to Grades 3 and 4 in the Heath-Edwards classification, such as concentric neointima and plexiform lesion were observed (FIG. 6(d)). The CD11c-Cre⁺Zc3h12a^{fl/fl} mice were also found to increase the right ventricular systolic pressure (RVSP) as compared with the control mice, demonstrating coincidence with the severity of pulmonary arterial occlusion (FIGS. 6(e) and 6(f)). Also in the CD11c-Cre⁺Zc3h12a^{fl/fl} mice, an age-dependent increase in the Fulton's coefficient (weight ratio of right ventricle/(left ventricle+septum)), which is an index of the degree of right ventricular hypertrophy, was observed (FIG. 6(g)). In the CD11c-Cre⁺Zc3h12a^{fl/fl} mice, also pulmonary vein occlusion, which is a hallmark of pulmonary vein occlusion (PVOD), was observed (FIG. 6(h)). These results teach that the CD11c-Cre⁺Zc3h12a^{fl/fl} mice spontaneously develop severe PAH relevant to CTD-PAH.

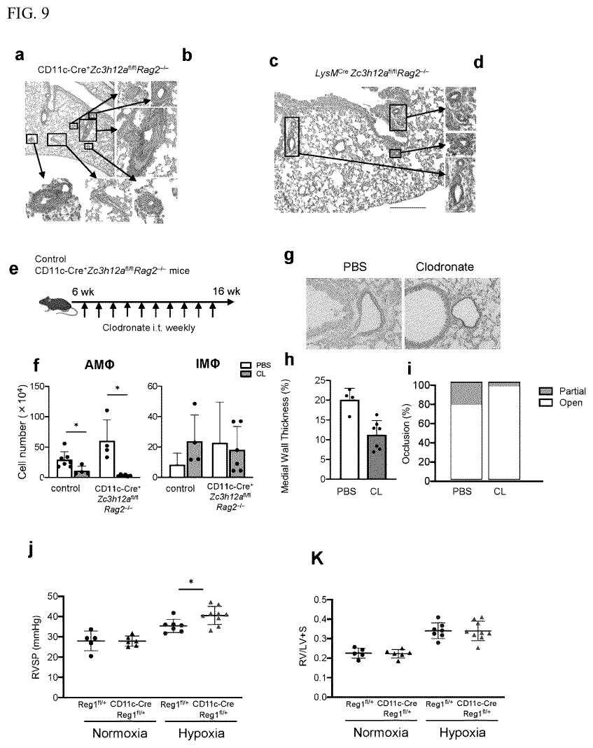

Since the CD11c-Cre⁺Zc3h12a^{fl/fl} mice lack Regnase-1 in alveolar macrophage and cDC, then influence of the Regnase-1 deficiency in these cells was examined. In the CD11c-Cre⁺Zc3h12a^{fl/fl} mice, cell counts of CD103⁺cDC1 and CD11b⁺cDC2 were found to increase, meanwhile the alveolar macrophage count was not found to increase (FIG. 7(a)). In cDC of the CD11c-Cre⁺Zc3h12a^{fl/fl} mice, expression of costimulatory molecules CD40 and CD80, and expression of cytokines such as Il1b and Il6 were however not found to increase (FIGS. 7(b) and 7(c)). On the other hand, the Regnase-1 deficiency was found to largely increase cytokine genes in the macrophage (FIG. 7(d)), and this taught that the alveolar macrophage deficient in Regnase-1 induces onset of PAH by way of inflammatory cytokines.

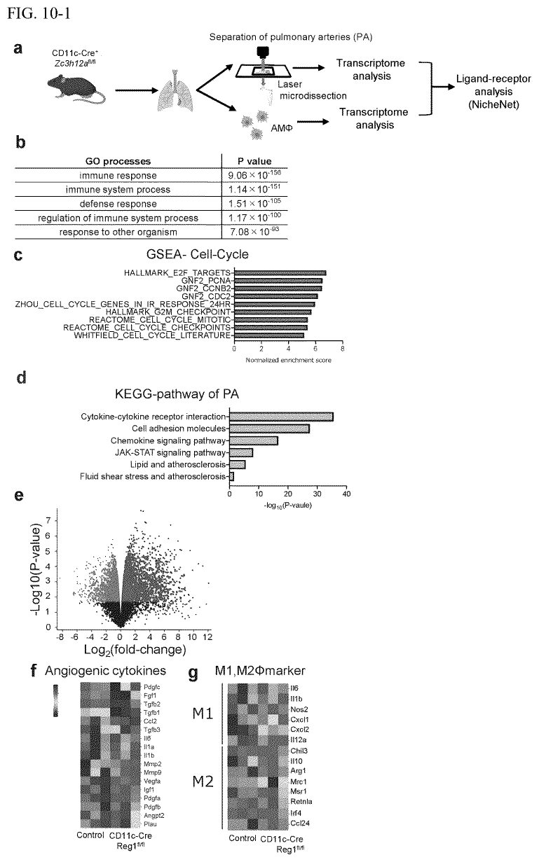

For the purpose of investigating the type of bone marrow cell involved in the onset of PAH induced by Regnase-1 deficiency, Lysm^{Cre/+}Zc3h12a^{fl/fl} mice deficient in Regnase-1 in lung macrophage and neutrophil were created (FIG. 8(a)). The Lysm^{Cre/+}Zc3h12a^{fl/fl} mice spontaneously developed severe PAH characterized by increased Fulton's coefficient and increased RVSP (FIGS. 6(i) and 6(j)). Also pathological changes that correspond to Grades 1 to 4 in the Heath-Edwards classification were observed in the lungs of the Lysm^{Cre/+}Zc3h12a^{fl/fl} mice (FIGS. 6(k) and 6(l)), and also in terms of PVOD characteristics (FIG. 6(m)). These results taught that the mice, deficient in Regnase-1 in macrophage and neutrophil but not in DC, were found to develop PAH relevant to CTD-PAH. Since both the CD11c-Cre⁺Zc3h12a^{fl/fl} mice and the Lysm^{Cre/+}Zc3h12a^{fl/fl} mice lack Regnase-1 in alveolar macrophage, the results taught that the mice deficient in Regnase-1 in alveolar macrophage spontaneously develop PAH.

### (3) Analysis of Effect of Regnase-1 Deficiency in Alveolar Macrophage on Progression of PAH

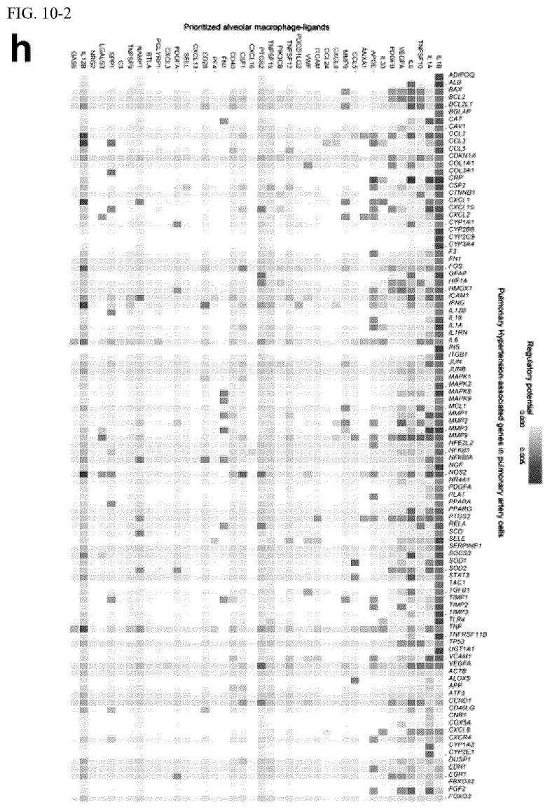

Not only innate immune cell, but also lymphocyte involved in acquired immunity are considered to be involved in the pathogenesis of PAH. In fact, Regnase-1 deficiency in the bone marrow cell of the CD11c-Cre⁺Zc3h12a^{fl/fl} mice or the Lysm^{Cre/+}Zc3h12a^{fl/fl} mice causes infiltration of acquired immunity such as T cell and B cell (FIG. 7(a)). For the purpose of examining whether or not the acquired immune cells are involved in pulmonary artery remodeling, these mice were crossed with Rag2 knockout mice lacking T cell and B cell (Rag2^{-/-} mice). The results taught that, in CD11c-Cre⁺Zc3h12a^{fl/fl} mice or Lysm^{Cre/+}Zc3h12a^{fl/fl} mice, severe pulmonary vascular remodeling accompanied by media thickening and vascular occlusion was observed with aging (FIGS. 9(a) to 9(d)), although considerably attenuated even by Rag2 deficiency, and it was found that also myeloid cells were directly involved in the pathogenesis, in addition to acquired immune cells. In order to further confirm the effect of alveolar macrophages in PAH with Regnase-1 deficiency, clodronate liposome was intratracheally administered to mice once a week to deplete the alveolar macrophage (FIG. 9(e)). From the results of RT-qPCR, it was confirmed that CD11c-Cre⁺Zc3h12a^{fl/fl}Rag2^{-/-} mice treated with the clodronate liposome depleted the alveolar macrophage, but did not deplete the interstitial macrophage (FIG. 9(f)). As seen in FIGS. 9(g) to 9(i), the clodronate liposome treatment significantly improved the media thickening and occlusion rate in CD11c-Cre⁺Zc3h12a^{fl/fl}Rag2^{-/-} mice. That is, it was confirmed also from these results that the Regnase-1 deficiency in alveolar macrophage can induce vascular remodeling to develop PAH.

(4) Effects of Chronic Hypoxic Load on Regnase-1 flox Heterozygous (Reg1 f/+) Mice and CD11c-Cre⁺Zc3h12a^{fl/+} Mice

The PH model mice ever established have PH induced by hypoxic exposure, or by Sugen treatment followed by hypoxic exposure. CD11c-Cre⁺Zc3h12a^{fl/+} mice were used to confirm whether or not Regnase-1 in bone marrow cell is involved in pathogenesis of hypoxia-induced PH in mice. Reg1 f/+ mice having Regnase-1 heterozygote in bone marrow cell did not spontaneously develop PH, meanwhile the CD11c-Cre⁺Zc3h12a^{fl/+} mice bred under hypoxic exposure demonstrated equivalent Fulton coefficient and increased RVSP, as compared with those of the control Zc3h12a^{fl/+} mice (FIGS. 9(j) and 9(k)). The results teach that Regnase-1 in the alveolar macrophage takes part in prevention of PH induced under hypoxic conditions.

(5) Analysis of Ligand-Target Binding between Regnase-1-Deficient Alveolar Macrophage and Pulmonary Artery

For the purpose of clarifying a mechanism of pulmonary artery remodeling induced by Regnase-1 deficient alveolar macrophage, conducted first was transcriptome analysis on pulmonary arteries prepared by laser microdissection (FIG. 10(a)). Gene ontology (GO) analysis revealed that, among the genes upregulated in the pulmonary artery of the CD11c-Cre⁺Zc3h12a^{fl/fl} mice, a gene group involved in the immune response was highly enriched (FIG. 10(b)). Also gene set enrichment analysis revealed that, among the genes upregulated in the pulmonary artery of the CD11c-Cre⁺Zc3h12a^{fl/fl} mice, a gene set involved in the cell cycle and cell proliferation was highly enriched (FIG. 10(b)). Also the KEGG pathway analysis revealed that genes in the cytokine-cytokine receptor signaling pathway were highly enriched in the pulmonary artery of the CD11c-Cre⁺Zc3h12a^{fl/fl} mice (FIG. 10(d)). Next, transcriptomic changes of the CD11c-Cre⁺Zc3h12a^{fl/fl} mice and the wild-type control mice were examined by RNA sequencing analysis (FIG. 10(e)). The results taught that various genes (Il6, Nos2, Chil3, Arg1, and Mrc1) involved in activation towards both of M1 and M2 macrophages, were found to cause upregulation (FIG. 10(f)). It was also found that various growth factors and cytokines that can act as angiogenic factors were found to be upregulated in alveolar macrophages of the CD11c-Cre⁺Zc3h12a^{fl/fl} mice (FIG. 10(g)).

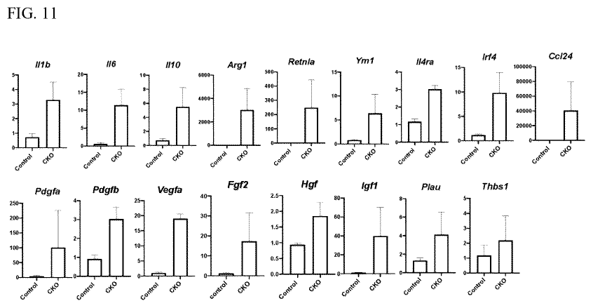

From these results, it is considered that, at least in part, a factor secreted by the Regnase-1 deficient alveolar macrophage can suppress proliferation of arterial smooth muscle cell and endothelial cell, thereby inducing vascular remodeling. Therefore, correlation of transcriptomic changes between alveolar macrophage and pulmonary artery was analyzed, by combining transcriptomic data of alveolar macrophage and pulmonary artery, with use of NicheNet which is a tool for predicting ligand-target links. The analysis successfully identified sets of ligand-target networks that potentially function between the Regnase-1 deficient alveolar macrophage and the pulmonary artery (FIGS. 10(h) and 10(i)). The ligands found to be particularly highly ranked include Il1a, Tnfsf10, Il6, Vegf, Pdgfb, Il33, Apoe, Anxa1, and Ccl5 (FIGS. 10(h) and 10(i)). In fact, the CD11c-Cre⁺Zc3h12a^{fl/fl} mice had increased expression levels of potential ligands in alveolar macrophage, as compared with the control mice (FIG. 11), suggesting that the cytokines and angiogenic factors produced by the Regnase-1 deficient alveolar macrophage induce proliferation of vascular endothelial cell and smooth muscle cell.

### (6) Functional Analysis of Regnase-1 in Alveolar Macrophage

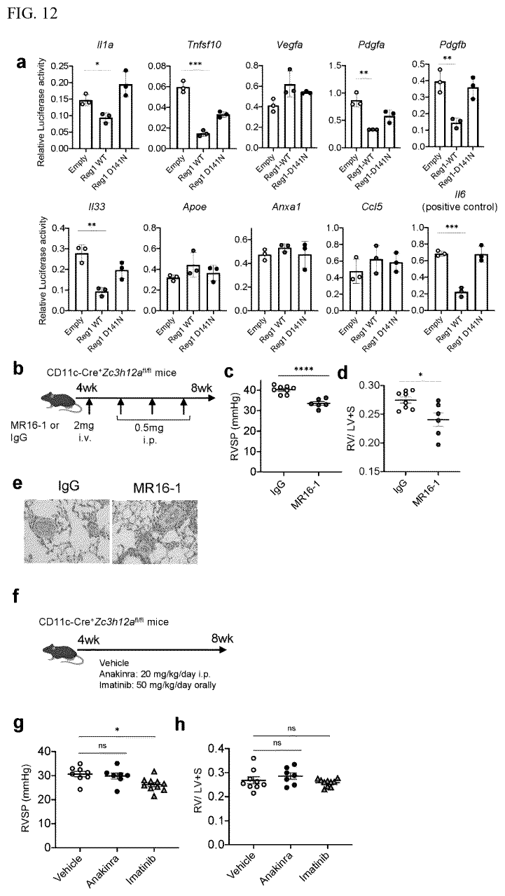

Next, whether or not the cytokines/angiogenic factors identified by the NicheNet method are directly regulated by mRNA decomposition mediated by Regnase-1, was analyzed. Since Regnase-1 recognizes the 3′UTR of a target mRNA, created herein was a luciferase reporter construct that includes Il1a, Tnfsf10, Il6, Vegf, Pdgfa, Pdgfb, Il33, Apoe, Anxa1, or Ccl5, besides the angiogenic factors Pdgf1, Egf, and Igf1. Overexpressed wild-type Regnase-1 (Reg1 WT) in HEK293T was found to significantly suppress the luciferase activity, not only in the presence of the 3′UTR of Il6, having been confirmed to be a target of Regnase-1, but also in the presence of the 3′UTRs of Il1a, Tnfsf10, Pdgfa, Pdgfb, and Il33 (FIG. 12(a)), meanwhile was not found to significantly suppress the luciferase activity in the presence of the 3′UTR of Pdgfa, Apoe, Anxa1, Ccl5, Egf, or Igf1. In a Regnase-1 (D141N) mutant with the nuclease activity inactivated, none of the tested genes could inhibit reporter activity (FIG. 12(b)). Regnase-1 was thus found to function as an RNase that controls these target mRNAs. These results clarified that Regnase-1 acts to decompose not only Il6 and Il1b, but also Il1a, Il33, Tnfsf10, and angiogenesis factors (Pdgfa, Pdgfb, etc.) in alveolar macrophage.

### (7) Effects of Administration of Anti-IL-6 Receptor Neutralizing Antibody on PAH Model Mice

Factors involved in the onset of PAH induced by Regnase-1 deficiency in alveolar macrophage were examined. IL-6 is an inflammatory cytokine that causes PAH, and is regulated by Regnase-1. Then, first, the role of IL-6 in the onset of PAH was examined, by treating the mice with MR16-1 or a control antibody (FIG. 12(b)). Blocking of IL-6 signal transmission in the CD11c-Cre⁺Zc3h12a^{fl/fl} mice, once a week for 5 weeks, was found to significantly suppress RVSP and Fulton coefficient (FIGS. 12(c) and 12(d)), proving that modulation of IL-6 mediated by Regnase-1 affects the onset of PAH. Histological analysis, however, demonstrated that the pulmonary artery occlusion in CD11c-Cre⁺Zc3h12a^{fl/fl} mice was alleviated, but was not completely healed after the MR16-1 treatment (FIG. 12(e)), suggesting that factors other than IL-6 may concurrently contribute to the pathogenesis of PAH.

### (8) Effects of Administration of PDGF Receptor Inhibitor and IL-1 Receptor Inhibitor on PAH Model Mice

For the purpose of clarifying an IL-6-independent mechanism involved in the onset of PAH induced by Regnase-1 deficiency in alveolar macrophage, the present disclosers focused on IL-1 and platelet-derived growth factor (PDGF) whose mRNAs are directly regulated by Regnase-1. IL-1-mediated response is inhibited also in vivo by treatment with an IL-1 receptor inhibitor (Anakinra), and PDGF signal transmission is inhibited by imatinib, which is a tyrosine kinase inhibitor that suppresses both PDGF receptors (PDGFRs)-α and -β. The CD11c-Cre⁺Zc3h12a^{fl/fl} mice failed in decreasing PVSP after daily intraperitoneal administration of anakinra, but significantly decreased RVSP after daily oral administration of imatinib (FIGS. 12(f) and 12(g)). The Fulton coefficient changed in response to imatinib, but only to an insignificant degree (FIG. 12(h)). These results teach that, although IL-6 is considered to hold the key for pathogenesis of PAH, also PDGF, but not IL-1, may be involved in the onset of PAH due to Regnase-1-deficient alveolar macrophage.

2-3. Suppression of Clinical Condition of PAH with Use of Morpholino Oligo (MO) that Suppresses Decomposition of Mouse Regnase-1

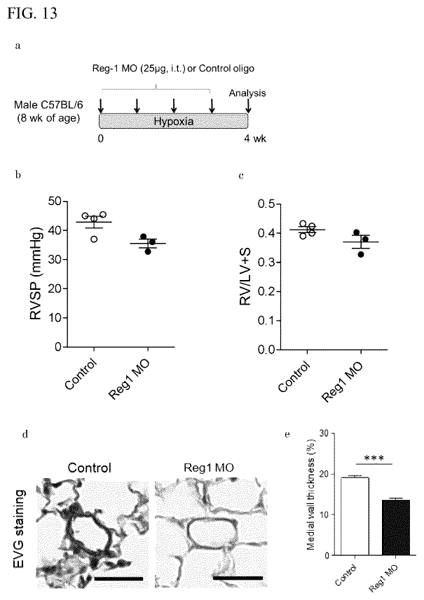

In the mice with administration of Regnase-1 morpholino oligo (Reg1MO) that suppresses decomposition of mouse Regnase-1, RVSP and the Fulton coefficient under hypoxia load were found to significantly increase, as compared with those in the mice with administration of the control oligo (FIGS. 13(b) and (c)). In the mice with administration of Reg1MO, also the degree of medial wall thickening in the pulmonary blood vessel was found to be significantly suppressed, as compared with the case where the control oligo was administered (FIGS. 13(d) and (e)). The results clarified that Regnase-1 mRNA may be suppressed from decomposing, through decomposition of the stem-loop structure in the 3′UTR of Regnase-1 mRNA, thereby successfully suppressing clinical condition of PAH.

1. A method for testing presence/absence, severity, or prognosis of pulmonary hypertension, the test method comprising:
measuring Regnase-1 gene expression level or Regnase-1 level in a sample isolated from a subject.

2. The method according to claim 1, wherein the pulmonary hypertension is pulmonary arterial hypertension.

3. The method according to claim 1, wherein the pulmonary hypertension is idiopathic pulmonary arterial hypertension, heritable pulmonary arterial hypertension, or pulmonary arterial hypertension associated with connective tissue disease.

4-16. (canceled)

17. A method for preventing or treating pulmonary hypertension, the method comprising a step of administering a substance that disrupts a stem-loop structure in 3′UTR of Regnase-1 mRNA, to a pulmonary hypertension patient or a person at risk of recurrence of pulmonary hypertension.

18-19. (canceled)

20. The method according to claim 17, wherein the substance is an oligonucleic acid that hybridizes to at least a part of a base sequence of a region that forms a stem of the stem-loop structure in 3′UTR of regnase-1 mRNA, and inhibits complementary binding in the stem-loop structure.

21. The method according to claim 17, wherein the substance is at least one oligonucleic acid selected from the group consisting of (a-1), (a-2), (b-1), and (b-2) below:
(a-1) an oligonucleic acid 10 to 30 bases in length that hybridizes to a sequence having 10 to 30 consecutive bases including positions 233 to 235 in SEQ ID NO: 1, within a base sequence from positions 206 to 242 in SEQ ID NO: 1;
(a-2) an oligonucleic acid 10 to 30 bases in length that hybridizes to a sequence having 10 to 30 consecutive bases including positions 241 to 243 in SEQ ID NO: 1, within a base sequence from positions 234 to 270 in SEQ ID NO: 1;
(b-1) an oligonucleic acid 10 to 30 bases in length that hybridizes to a sequence having 10 to 30 consecutive bases including positions 426 to 428 in SEQ ID NO: 1, within a base sequence from positions 399 to 439 in SEQ ID NO: 1; and
(b-2) an oligonucleic acid 10 to 30 bases in length that hybridizes to a sequence having a 10 to 30 consecutive bases including positions 438 to 440 in SEQ ID NO: 1, within a base sequence from positions 427 to 467 in SEQ ID NO: 1.

22. The method according to claim 21, wherein the substance comprises a combination of at least one oligonucleic acid selected from the group consisting of (a-1) and (a-2), and at least one oligonucleic acid selected from the group consisting of (b-1) and (b-2).

23. The method according to claim 17, wherein the pulmonary hypertension is pulmonary arterial hypertension.

24. The method according to claim 17, wherein the pulmonary hypertension is idiopathic pulmonary arterial hypertension, heritable pulmonary arterial hypertension, or pulmonary arterial hypertension associated with connective tissue disease.


SEQUENCE LISTING (19 sequences)

SEQ ID NO: 1 - RNA, 765 nt, Homo sapiens
  source 1..765: mol_type = mRNA
  3'UTR 1..765: gene = ZC3H12A
gctgcctgtggctggcaagggcagcacccccagcctccaagggccgtcaggctgggctttgggccattgagcagcccattcccagccctgaggcccaccccagaggctggacagagggaggattcaagtcgggaaggaaacccacaaaccaaagatactgtaggattggttctggcccatgcagcacctctagctgtctgcctcagtgggtcagaagcgatcaccctgttgatacacattgtatctctgtagtttaaggagacgctgccggtaacggcgtcggtccgtggctgaggcccaaaccgtcttttctctcagagggtggggagggaggtgggggcagcagaggcctgggctgggtgccctgtgcacgccaccccacttccgccctacccctgggacgttggccttggctggctagttgggcaccgtgtgcctgccctccaagggcctcctctacgccaatgaggcctcatctgtgctctcgctgggcacgtggcttcatgtcagtaagcaagatgcttcttaataacccaccttctgccccactctattccttatcctgctgcccctgtaggggtcaagggccctccgtctacaccctcttcttctcctccatcctttattcagagtcatctcgcccttccccatgggtgggggaacctgtgtttgtttgtgtgcacatgtaaattttaaatattttaagcagaaagtccttacctcctgtaacacatcaataaagtacaatcattgtgagccctttca

SEQ ID NO: 2 - RNA, 865 nt, Mus musculus
  source 1..865: mol_type = mRNA
  3'UTR 1..865: gene = zc3h12a
gccagaaggtggcgcaaggggctgtcgggcatcacagatagcggtccccagcccctgcctggcctaccctggaagctggacagaaggacaagtgaagcaggcaagggaatcctcaaaccaaagataccataggattggttctggccccgcggcaccccaacccgtcccgcacaggtcagaagtgatcaccctgttgatacacattgtatctctgtcatttaaggagacgctgccggtcaggccgtccatccgtggctgatgcccaaaccctcttttttttttctcagagggccgggtgggaggcatgggggagcagaggcctgagctggaccccaccttccaccctgtccctgggacgccggcaccaccggctagttaggcaccatgtgcctgctctctgaggccccctcaagccaatgcggcctcatccctgttcacagggcatgaggcttcatgttagtaagcaagatgcttctttaagcccctcccctgcccgctctgtccacctacacaccccccccccaaccagggctccaaggccctctgtttccacacctcccatgggtgggaggacacatgtatgctgtgtacagaggcgagatttaaatattttaaatgaaaaaggttgacaaaataaaggctattgccaggcaggctggagagatggctcagtggttaagagcaccgactgctcttctgaaggtcctgagttcaaatgtcagcaaccacatggtggctcacaaacatctgtgatgagatctggtgccctcttctggggtgtctgaagacagctacagtgtacttacatacaataataaatcttttaaaaaagagagaaatttaaaagaaaaaaaaagctattgcc

SEQ ID NO: 3 - protein, 599 aa, Homo sapiens
MSGPCGEKPVLEASPTMSLWEFEDSHSRQGTPRPGQELAAEEASALELQMKVDFFRKLGYSSTEIHSVLQKLGVQADTNTVLGELVKHGTATERERQTSPDPCPQLPLVPRGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLEHRKQPCPYGRKCTYGIKCRFFHPERPSCPQRSVADELRANALLSPPRAPSKDKNGRRPSPSSQSSSLLTESEQCSLDGKKLGAQASPGSRQEGLTQTYAPSGRSLAPSGGSGSSFGPTDWLPQTLDSLPYVSQDCLDSGIGSLESQMSELWGVRGGGPGEPGPPRAPYTGYSPYGSELPATAAFSAFGRAMGAGHFSVPADYPPAPPAFPPREYWSEPYPLPPPTSVLQEPPVQSPGAGRSPWGRAGSLAKEQASVYTKLCGVFPPHLVEAVMGRFPQLLDPQQLAAEILSYKSQHPSE

SEQ ID NO: 4 - protein, 596 aa, Mus musculus
MSDPCGTKPVQESNPTMSLWSLEDRHSSQGRPQPDQDPVAKEAPTSELQMKVDFFRKLGYSSSEIHSVLQKLGVQADTNTVLGELVKHGSATERECQALTAPSPQPPLVPRGGSTPKPSTLEPSLPEEDREGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAFESDGVVVSNDTYRDLQGERQEWKRFIEERLLMYSFVNDKFMPPDDPLGRHGPSLDNFLRKKPLPSEHRKQPCPYGKKCTYGIKCRFFHPERPSRPQRSVADELRANALLSPPRTPVKDKSSQRPSPASQSSSVSLEAEPGSLDGKKLGARSSPGPHREGSPQTCAPAGRSLPVSGGSFGPTEWLAHTQDSLPYTSQECLDSGIGSLESQMSELWGVRGGSPGESGPTRGPYAGYHSYGSKVPAAPSFSPFRPAMGAGHFSVPTDYVPPPPTYPSREYWSEPYPLPPPTPVLQEPQRPSPGAGGGPWGRVGDLAKERAGVYTKLCGVFPPHLVEAVMRRFPQLLDPQQLAAEILSYKSQHLSE

SEQ ID NO: 5 - RNA, 262 nt, Homo sapiens
  source 1..262: mol_type = mRNA
  3'UTR 1..262: gene = ZC3H12A
gtgggtcagaagcgatcaccctgttgatacacattgtatctctgtagtttaaggagacgctgccggtaacggcgtcggtccgtggctgaggcccaaaccgtcttttctctcagagggtggggagggaggtgggggcagcagaggcctgggctgggtgccctgtgcacgccaccccacttccgccctacccctgggacgttggccttggctggctagttgggcaccgtgtgcctgccctccaagggcctcctctacgccaatg

SEQ ID NO: 6 - RNA, 246 nt, Mus musculus
  source 1..246: mol_type = mRNA
  3'UTR 1..246: gene = zc3h12a
acaggtcagaagtgatcaccctgttgatacacattgtatctctgtcatttaaggagacgctgccggtcaggccgtccatccgtggctgatgcccaaaccctcttttttttttctcagagggccgggtgggaggcatgggggagcagaggcctgagctggaccccaccttccaccctgtccctgggacgccggcaccaccggctagttaggcaccatgtgcctgctctctgaggccccctcaagcca

SEQ ID NO: 7 - DNA, 23 bp, synthetic construct
  misc_feature 1..23: note = Morpholino oligonucleotide for human Regnase-1
  source 1..23: mol_type = other DNA
aatgtgtatcaacagggtgatcg

SEQ ID NO: 8 - DNA, 23 bp, synthetic construct
  misc_feature 1..23: note = Morpholino oligonucleotide for human Regnase-1
  source 1..23: mol_type = other DNA
cttaaactacagagatacaatgt

SEQ ID NO: 9 - DNA, 19 bp, synthetic construct
  misc_feature 1..19: note = Morpholino oligonucleotide for human Regnase-1
  source 1..19: mol_type = other DNA
acggtgcccaactagccag

SEQ ID NO: 10 - DNA, 19 bp, synthetic construct
  misc_feature 1..19: note = Morpholino oligonucleotide for human Regnase-1
  source 1..19: mol_type = other DNA
ggcccttggagggcaggca

SEQ ID NO: 11 - DNA, 23 bp, synthetic construct
  misc_feature 1..23: note = Morpholino oligonucleotide for mouse Regnase-1
  source 1..23: mol_type = other DNA
aatgtgtatcaacagggtgatca

SEQ ID NO: 12 - DNA, 23 bp, synthetic construct
  misc_feature 1..23: note = Morpholino oligonucleotide for mouse Regnase-1
  source 1..23: mol_type = other DNA
cttaaatgacagagatacaatgt

SEQ ID NO: 13 - DNA, 20 bp, synthetic construct
  misc_feature 1..20: note = Morpholino oligonucleotide for mouse Regnase-1
  source 1..20: mol_type = other DNA
atggtgcctaactagccggt

SEQ ID NO: 14 - DNA, 20 bp, synthetic construct
  misc_feature 1..20: note = Morpholino oligonucleotide for mouse Regnase-1
  source 1..20: mol_type = other DNA
cctcagagagcaggcacatg

SEQ ID NO: 15 - DNA, 20 bp, synthetic construct
  misc_feature 1..20: note = Fw primer for Regnase-1
  source 1..20: mol_type = other DNA
gaagaggaaaaggagggcag

SEQ ID NO: 16 - DNA, 20 bp, synthetic construct
  misc_feature 1..20: note = Rv primer for Regnase-1
  source 1..20: mol_type = other DNA
ctccaggatggcacaaacac

SEQ ID NO: 17 - DNA, 19 bp, synthetic construct
  misc_feature 1..19: note = Fw primer for hGAPDH
  source 1..19: mol_type = other DNA
atggggaaggtgaaggtcg

SEQ ID NO: 18 - DNA, 22 bp, synthetic construct
  misc_feature 1..22: note = Rv primer for hGAPDH
  source 1..22: mol_type = other DNA
ggggtcattgatggcaacaata

SEQ ID NO: 19 - DNA, 25 bp, synthetic construct
  misc_feature 1..25: note = Control oligo
  source 1..25: mol_type = other DNA
cctcttacctcagttacaatttata